(19) United States
(12) Patent Application Publication
(10) Pub. No.: US 2019/0192488 A1
(43) Pub. Date: Jun. 27, 2019

(54) USE OF MITOCHONDRIAL ACTIVITY INHIBITORS FOR THE TREATMENT OF POOR PROGNOSIS ACUTE MYELOID LEUKEMIA

(71) Applicant: UNIVERSITÉ DE MONTRÉAL, Montreal (CA)

(72) Inventors: Guy SAUVAGEAU, Montréal (CA); Irène BACCELLI, Montréal (CA)

(73) Assignee: Université de Montréal, Montreal, QC (CA)

(21) Appl. No.: 16/322,821

(22) PCT Filed: Aug. 1, 2017
(86) PCT No.: PCT/CA2017/050921

§ 371 Date: Feb. 1, 2019

(30) Foreign Application Priority Data
Aug. 2, 2016 (CA) 2,937,896

(51) Int. Cl. A61K 31/422; A61P 35/02

(52) U.S. Cl. CPC A61K 31/422; A61P 35/02

(57) ABSTRACT

A method for treating acute myeloid leukemia (AML), such as poor risk AML, by administering to a subject in need en thereof an effective amount of a mitochondrial activity inhibitor, for example a class A electron transport chain (ETC) complex I inhibitor such as Mubritinib or a pharmaceutically acceptable salt thereof, is disclosed. The AML to be treated may be characterized by certain features, such as high level of expression of one or more Homeobox (HOX)-network genes, high and/or low expression of specific genes, the presence of one or more cytogenetic or molecular risk factors such as intermediate cytogenetic risk, Normal Karyotype (NK), mutated NPM1, mutated CEBPA, mutated FLT3, mutated DNMT3A, mutated TET2, mutated IDH1, mutated IDH2, mutated RUNX1, mutated WT 1, mutated SRSF2, intermediate cytogenetic risk with abnormal karyotype (intern(abnK)), trisomy 8 (+8) and/or abnormal chromosome (5/7), and/or a high leukemic stem cell (LSC) frequency.

### CROSS-REFERENCE TO RELATED APPLICATIONS

The present application claims the benefit of Canadian patent application serial No. 2,937,896, filed on Aug. 2, 2016, which is incorporated herein by reference in its entirety.

### TECHNICAL FIELD

The present disclosure generally relates to the treatment of acute myeloid leukemia (AML), and more specifically of AML subtypes typically associated with poor prognosis.

### BACKGROUND ART

Acute Myeloid Leukemia (AML) is a particularly lethal form of cancer, with most patients dying within two years of diagnosis. It is one of the leading causes of death among young adults. AML is a collection of neoplasms with heterogeneous pathophysiology, genetics and prognosis. Mainly based on cytogenetics and molecular analysis, AML patients are presently classified into groups or subsets of AML with markedly contrasting prognosis. Approximately 45% of all AML patients are currently classified into distinct groups with variable prognosis based on the presence or absence of specific recurrent cytogenetic abnormalities.

Targeting FLT3 receptor tyrosine kinases with FLT3 tyrosine kinase inhibitors (TKIs) has shown encouraging results in the treatment of FLT3-mutated AML (generally associated with poor clinical outcome), but in most patients, responses are incomplete and not sustained. Also, the induction of acquired resistance to TKIs has emerged as a clinical problem. Also, inhibitors of other pathologically activated kinases in AML such as c-KIT and JAK2 have achieved only rare bone marrow responses.

There is thus a need for the identification of novel therapeutic strategies for the treatment of AML, such as AML associated with poor prognosis.

The present description refers to a number of documents, the content of which is herein incorporated by reference in their entirety.

### SUMMARY OF THE INVENTION

The present disclosure provides the following items 1 to 88:

1. Use of a mitochondrial activity inhibitor, preferably a class A complex I ETC inhibitor, more preferably Mubritinib or a pharmaceutically acceptable salt thereof, for treating acute myeloid leukemia (AML) in a subject.
2. Use of a mitochondrial activity inhibitor, preferably a class A complex I ETC inhibitor, more preferably Mubritinib or a pharmaceutically acceptable salt thereof, for the manufacture of a medicament for treating acute myeloid leukemia (AML) in a subject.
3. The use of item 1 or 2, wherein said AML is poor prognosis AML.
4. The use of any one of items 1 to 3, wherein said AML comprises at least one of the following features: (a) high level of expression of one or more homeobox (HOX)-network genes; (b) high level of expression of one or more of the genes depicted in Table 1; (c) low level of expression of one or more of the genes depicted in Table 2; (d) one or more of the following cytogenetic or molecular risk factor: intermediate cytogenetic risk, Normal Karyotype (NK), mutated NPM1, mutated CEBPA, mutated FLT3, mutated DNA methylation genes, mutated RUNX, mutated WT1, mutated SRSF2, intermediate cytogenetic risk with abnormal karyotype (intern(abnK)), trisomy 8 (+8) and abnormal chr(5/7); and (e) a leukemic stem cell (LSC) frequency of about 1 LSC per 1×10⁶ total cells, or more.
5. The use of item 4, wherein said AML comprises high level of expression of one or more HOX-network genes.
6. The use of item 5, wherein said one or more HOX-network genes are HOXB1, HOXB2, HOXB3, HOXB5, HOXB6, HOXB7, HOXB9, HOXB-AS3, HOXA1, HOXA2, HOXA3, HOXA4, HOXA5, HOXA6, HOXA7, HOXA9, HOXA10, HOXA10-AS, HOXA11, HOXA11-AS, MEIS1 and/or PBX3.
7. The use of item 6, wherein said one or more HOX-network genes are HOXA9 and/or HOXA10.
8. The use of any one of items 4-7, wherein said AML comprises high level of expression of one or more of the genes depicted in Table 1.
9. The use of any one of items 4-8, wherein said AML comprises low level of expression of one or more of the genes depicted in Table 2.
10. The use of any one of items 4-9, wherein said AML comprises one or more of the cytogenetic or molecular risk factor defined in item (d) of item 1.
11. The use of item 10, wherein said AML is intermediate cytogenetic risk AML and/or NK-AML
12. The use of item 10 or 11, wherein said AML comprises at least two of said cytogenetic or molecular risk factors.
13. The use of item 12, wherein said AML comprises at least three of said cytogenetic or molecular risk factors.
14. The use of any one of items 4 to 13, wherein said AML comprises a mutated NPM1, a mutated FLT3 and/or a mutated DNA methylation gene.
15. The use of item 14, wherein said DNA methylation gene is DNMT3A or IDH1.
16. The use of item 14 or 15, wherein said AML comprises a mutated NPM1, a mutated FLT3 and a mutated DNA methylation gene, preferably the DNA methylation gene is DNMT3A.
17. The use of any one of items 4 to 16, wherein said mutated FLT3 is FLT3 with Internal tandem duplication (FLT3-ITD).
18. The use of any one of items 4 to 17, wherein said AML comprises an LSC frequency of about 1 LSC per 1×10⁶ total cells, or more.
19. The use of item 18, wherein said AML comprises an LSC frequency of about 1 LSC per 5×10⁵ total cells, or more.
20. The use of any one of items 4 to 19, wherein said AML comprises at least two of features (a) to (e).
21. The use of item 20, wherein said AML comprises at least three of features (a) to (e).
22. The use of any one of items 4 to 21, wherein said AML is NK-AML with mutated NPM1.
23. The use of any one of items 1 to 22, wherein the mitochondrial activity inhibitor is present in a pharmaceutical composition.
24. The use of any one of items 1 to 23, wherein the subject is a pediatric subject.
25. The use of any one of items 1 to 23, wherein the subject is an adult subject.
26. A mitochondrial activity inhibitor, preferably a class A complex I ETC inhibitor, more preferably Mubritinib or a pharmaceutically acceptable salt thereof, for use in the treatment of acute myeloid leukemia (AML).
27. The mitochondrial activity inhibitor, preferably the class A complex I ETC inhibitor, more preferably Mubritinib or a pharmaceutically acceptable salt thereof, for use according to item 26, wherein said AML is poor prognosis AML.
28. The mitochondrial activity inhibitor, preferably the class A complex I ETC inhibitor, more preferably Mubritinib or a pharmaceutically acceptable salt thereof, for use according to item 26 or 27, wherein said AML comprises at least one of the following features: (a) high level of expression of one or more homeobox (HOX)-network genes; (b) high level of expression of one or more of the genes depicted in Table 1; (c) low level of expression of one or more of the genes depicted in Table 2; (d) one or more of the following cytogenetic or molecular risk factor: intermediate cytogenetic risk, Normal Karyotype (NK), mutated NPM1, mutated CEBPA, mutated FLT3, mutated DNA methylation genes, mutated RUNX1, mutated WT1, mutated SRSF2, intermediate cytogenetic risk with abnormal karyotype (intern(abnK)), trisomy 8 (+8) and abnormal chr(5/7); and (e) a leukemic stem cell (LSC) frequency of about 1 LSC per 1×10⁶ total cells, or more.
29. The mitochondrial activity inhibitor, preferably the class A complex I ETC inhibitor, more preferably Mubritinib or a pharmaceutically acceptable salt thereof, for use according to item 28, wherein said AML comprises high level of expression of one or more HOX-network genes.
30. The mitochondrial activity inhibitor, preferably the class A complex I ETC inhibitor, more preferably Mubritinib or a pharmaceutically acceptable salt thereof, for use according to item 29, wherein said one or more HOX-network genes are HOXB1, HOXB2, HOXB3, HOXB5, HOXB6, HOXB7, HOXB9, HOXB-AS3, HOXA1, HOXA2, HOXA3, HOXA4, HOXA5, HOXA6, HOXA7, HOXA9, HOXA10, HOXA10-AS, HOXA11, HOXA11-AS, MEIS1 and/or PBX3.
31. The mitochondrial activity inhibitor, preferably the class A complex I ETC inhibitor, more preferably Mubritinib or a pharmaceutically acceptable salt thereof, for use according to item 30, wherein said one or more HOX-network genes are HOXA9 and/or HOXA10.
32. The mitochondrial activity inhibitor, preferably the class A complex I ETC inhibitor, more preferably Mubritinib or a pharmaceutically acceptable salt thereof, for use according to any one of items 28-31, wherein said AML comprises high level of expression of one or more of the genes depicted in Table 1.
33. The mitochondrial activity inhibitor, preferably the class A complex I ETC inhibitor, more preferably Mubritinib or a pharmaceutically acceptable salt thereof, for use according to any one of items 28-32, wherein said AML comprises low level of expression of one or more of the genes depicted in Table 2.
34. The mitochondrial activity inhibitor, preferably the class A complex I ETC inhibitor, more preferably Mubritinib or a pharmaceutically acceptable salt thereof, for use according to any one of items 28-33, wherein said AML comprises one or more of the cytogenetic or molecular risk factor defined in item (d) of item 28.
35. The mitochondrial activity inhibitor, preferably the class A complex I ETC inhibitor, more preferably Mubritinib or a pharmaceutically acceptable salt thereof, for use according to item 34, wherein said AML is intermediate cytogenetic risk AML and/or NK-AML.
36. The mitochondrial activity inhibitor, preferably the class A complex I ETC inhibitor, more preferably Mubritinib or a pharmaceutically acceptable salt thereof, for use according to any one of items 28-35, wherein said AML comprises at least two of said cytogenetic or molecular risk factors.
37. The mitochondrial activity inhibitor, preferably the class A complex I ETC inhibitor, more preferably Mubritinib or a pharmaceutically acceptable salt thereof, for use according to item 36, wherein said AML comprises at least three of said cytogenetic or molecular risk factors.
38. The mitochondrial activity inhibitor, preferably the class A complex I ETC inhibitor, more preferably Mubritinib or a pharmaceutically acceptable salt thereof, for use according to any one of items 28-37, wherein said AML comprises a mutated NPM1, a mutated FLT3 and/or a mutated DNA methylation gene.
39. The mitochondrial activity inhibitor, preferably the class A complex I ETC inhibitor, more preferably Mubritinib or a pharmaceutically acceptable salt thereof, for use according to item 38, wherein said DNA methylation gene is DNMT3A or IDH1.
40. The mitochondrial activity inhibitor, preferably the class A complex I ETC inhibitor, more preferably Mubritinib or a pharmaceutically acceptable salt thereof, for use according to item 38 or 39, wherein said AML comprises a mutated NPM1, a mutated FLT3 and a mutated DNA methylation gene, preferably the DNA methylation gene is DNMT3A.
41. The mitochondrial activity inhibitor, preferably the class A complex I ETC inhibitor, more preferably Mubritinib or a pharmaceutically acceptable salt thereof, for use according to any one of items 28 to 40, wherein said mutated FLT3 is FLT3 with Internal tandem duplication (FLT3-ITD).
42. The mitochondrial activity inhibitor, preferably the class A complex I ETC inhibitor, more preferably Mubritinib or a pharmaceutically acceptable salt thereof, for use according to any one of items 28 to 41, wherein said AML comprises an LSC frequency of about 1 LSC per 1×10⁶ total cells, or more.
43. The mitochondrial activity inhibitor, preferably the class A complex I ETC inhibitor, more preferably Mubritinib or a pharmaceutically acceptable salt thereof, for use according to item 42, wherein said AML comprises an LSC frequency of about 1 LSC per 5×10⁵ total cells, or more.
44. The mitochondrial activity inhibitor, preferably the class A complex I ETC inhibitor, more preferably Mubritinib or a pharmaceutically acceptable salt thereof, for use according to any one of items 28 to 43, wherein said AML comprises at least two of features (a) to (e) defined in item 28.
45. The mitochondrial activity inhibitor, preferably the class A complex I ETC inhibitor, more preferably Mubritinib or a pharmaceutically acceptable salt thereof, for use according to item 44, wherein said AML comprises at least three of features (a) to (e) defined in item 28.
46. The mitochondrial activity inhibitor, preferably the class A complex I ETC inhibitor, more preferably Mubritinib or a pharmaceutically acceptable salt thereof, for use according to any one of items 28 to 45, wherein said AML is NK-AML with mutated NPM1.
47. The mitochondrial activity inhibitor, preferably the class A complex I ETC inhibitor, more preferably Mubritinib or a pharmaceutically acceptable salt thereof, for use according to any one of items 26 to 46, wherein the mitochondrial activity inhibitor is present in a pharmaceutical composition.
48. The mitochondrial activity inhibitor, preferably the class A complex I ETC inhibitor, more preferably Mubritinib or a pharmaceutically acceptable salt thereof, for use according to any one of items 26 to 47, wherein the subject is a pediatric subject.
49. The mitochondrial activity inhibitor, preferably the class A complex I ETC inhibitor, more preferably Mubritinib or a pharmaceutically acceptable salt thereof, for use according to any one of items 26 to 47, wherein the subject is an adult subject.
50. A method for determining the likelihood that a subject suffering from acute myeloid leukemia (AML) responds to a treatment with a mitochondrial activity inhibitor, preferably the class A complex I ETC inhibitor, more preferably Mubritinib or a pharmaceutically acceptable salt thereof, said method comprising determining whether AML cells from said subject comprise at least one of the following features: (a) high level of expression of one or more homeobox (HOX)-network genes; (b) high level of expression of one or more of the genes depicted in Table 1; (c) low level of expression of one or more of the genes depicted in Table 2; (d) one or more of the following cytogenetic or molecular risk factor: intermediate cytogenetic risk, Normal Karyotype (NK), mutated NPM1, mutated CEBPA, mutated FLT3, mutated DNA methylation genes, mutated RUNX1, mutated WT1, mutated SRSF2, intermediate cytogenetic risk with abnormal karyotype (intern(abnK)), trisomy 8 (+8) and abnormal chr(5/7); and (e) a leukemic stem cell (LSC) frequency of about 1 LSC per 1×10⁶ total cells, or more, wherein the presence of said at least one of the following features in said AML cells is indicative that the subject has a high likelihood of responding to said treatment.
51. The method of item 50, wherein said AML cells express high level of expression of one or more HOX-network genes.
52. The method of item 51, wherein said one or more HOX-network genes are HOXB1, HOXB2, HOXB3, HOXB5, HOXB6, HOXB7, HOXB9, HOXB-AS3, HOXA1, HOXA2, HOXA3, HOXA4, HOXA5, HOXA6, HOXA7, HOXA9, HOXA10, HOXA10-AS, HOXA11, HOXA11-AS, MEIS1 and/or PBX3.
53. The method of item 52, wherein said one or more HOX-network genes are HOXA9 and/or HOXA10.
54. The method of any one of items 50-53, wherein said AML cells express high level of expression of one or more of the genes depicted in Table 1.
55. The method of any one of items 50-55, wherein said AML cells express low level of expression of one or more of the genes depicted in Table 2.
56. The method of any one of items 50-55, wherein said AML cells comprise one or more of the cytogenetic or molecular risk factor defined in item (d) of item 50.
57. The method of any one of items 50-56, wherein said AML is intermediate cytogenetic risk AML and/or NK-AML.
58. The method of any one of items 50-57, wherein said AML cells comprise at least two of said cytogenetic or molecular risk factors.
59. The method of item 58, wherein said AML cells comprise at least three of said cytogenetic or molecular risk factors.
60. The method of item 58, wherein said AML comprises a mutated NPM1, a mutated FLT3 and/or a mutated DNA methylation gene.
61. The method of item 60, wherein said DNA methylation gene is DNMT3A or IDH1
62. The method of item 60 or 61, wherein said AML cells comprise a mutated NPM1, a mutated FLT3 and a mutated DNA methylation gene, preferably the DNA methylation gene is DNMT3A.
63. The method of any one of items 50 to 62, wherein said mutated FLT3 is FLT3 with Internal tandem duplication (FLT3-ITD).
64. The method of any one of items 50 to 63, wherein the LSC frequency in said AML cells is about 1 LSC per 1×10⁶ total cells, or more.
65. The method of item 64, wherein the LSC frequency in said AML cells is about 1 LSC per 5×10⁵ total cells, or more.
66. The method of any one of items 50 to 65, wherein said AML is NK-AML with mutated NPM1.
67. A method for treating acute myeloid leukemia (AML), said method comprising administering to a subject in need thereof an effective amount of a mitochondrial activity inhibitor, preferably the class A complex I ETC inhibitor, more preferably Mubritinib or a pharmaceutically acceptable salt thereof.
68. The method of item 67, wherein said AML is poor prognosis AML.
69. The method of item 67 or 68, wherein said AML comprises at least one of the following features: (a) high level of expression of one or more homeobox (HOX)-network genes; (b) high level of expression of one or more of the genes depicted in Table 1; (c) low level of expression of one or more of the genes depicted in Table 2; (d) one or more of the following cytogenetic or molecular risk factor: intermediate cytogenetic risk, Normal Karyotype (NK), mutated NPM1, mutated CEBPA, mutated FLT3, mutated DNA methylation genes, mutated RUNX1, mutated WT1, mutated SRSF2, intermediate cytogenetic risk with abnormal karyotype (intern(abnK)), trisomy 8 (+8) and abnormal chr(5/7); and (e) a leukemic stem cell (LSC) frequency of about 1 LSC per 1×10⁶ total cells, or more.
70. The method of item 69, wherein said AML comprises high level of expression of one or more HOX-network genes.
71. The method of item 69 or 70, wherein said one or more HOX-network genes are HOXB1, HOXB2, HOXB3, HOXB5, HOXB6, HOXB7, HOXB9, HOXB-AS3, HOXA1, HOXA2, HOXA3, HOXA4, HOXA5, HOXA6, HOXA7, HOXA9, HOXA10, HOXA10-AS, HOXA11, HOXA11-AS, MEIS1 and/or PBX3.
72. The method of item 71, wherein said one or more HOX-network genes are HOXA9 and/or HOXA10.
73. The method of any one of items 69-72, wherein said AML comprises high level of expression of one or more of the genes depicted in Table 1.
74. The method of any one of items 69-73, wherein said AML comprises low level of expression of one or more of the genes depicted in Table 2.
75. The method of any one of items 69-74, wherein said AML comprises one or more of the cytogenetic or molecular risk factor defined in item (d) of item 69.
76. The method of any one of items 69-75, wherein said AML is intermediate cytogenetic risk AML and/or NK-AML.
77. The method of any one of items 69-76, wherein said AML comprises at least two of said cytogenetic or molecular risk factors.
78. The method of item 77, wherein said AML comprises at least three of said cytogenetic or molecular risk factors.
79. The method of item 77, wherein said AML comprises a mutated NPM1, a mutated FLT3 and/or a mutated DNA methylation gene.
80. The method of item 80, wherein said AML comprises a mutated NPM1, a mutated FLT3 and a mutated DNA methylation gene.
81. The method of item 79 or 80, wherein said DNA methylation gene is DNMT3A or IDH1, preferably DNMT3A.
82. The method of any one of items 69-81, wherein said mutated FLT3 is FLT3 with Internal tandem duplication (FLT3-ITD).
83. The method of any one of items 69-82, wherein said AML comprises an LSC frequency of about 1 LSC per 1×10⁶ total cells, or more.
84. The method of item 83, wherein said AML comprises an LSC frequency of about 1 LSC per 5×10⁵ total cells, or more.
85. The method of any one of items 69-84, wherein said AML is NK-AML with mutated NPM1.
86. The method of any one of items 67-85, wherein the mitochondrial activity inhibitor is present in a pharmaceutical composition.
87. The method of any one of items 67-86, wherein the subject is a pediatric subject.
88. The method of any one of items 67-86, wherein the subject is an adult subject.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A-D show the link between expression of HOX-network genes and AML prognosis. FIG. 1A: Identification of patient samples with consistent high expression of HOX-network gene members (black, upper right of each graph) and with consistent low expression of HOX-network gene members (grey, lower left of each graph) in the prognostic Leucegene AML cohort (263 AML patients) studied herein, a cohort of AML patients for which sufficient information is available to make survival studies. Values in brackets indicate the range of expression of each gene. FIG. 1B: Overall survival of AML patients belonging to HOX-network high (lower line, n=100) versus low (upper line, n=27) subgroups. FIG. 1C: Identification of a HOXA9/HOXA10 high AML sample population. FIG. 1D: Overall survival of AML patients belonging to the HOXA9/HOXA10 high group (lower line, n=131) and HOXA9/HOXA10 med/low group (upper line, n=132). In FIG. 1B and FIG. 1D, p-values were determined by log-rank test. Abbreviations: AML: Acute myeloid leukemia; HOX: Homeobox; MEIS1: MEIS Homeobox 1; RPKM: Reads Per Kilobase per Million mapped reads; Std: standard deviation.

FIGS. 2A-I show the characterization of the pharmacological response of HOX-high AML cells. FIG. 2A: Overview of the primary screen workflow of 60 compounds (Table 4). HOXA9 and HOXA10 were used as representative genes for the distinction between HOX-high (dark grey, n=131) versus HOX-med/low (light grey, n=132) patient samples. FIG. 2B: Summary of the results of the primary screen leading to the identification of Mubritinib as a candidate drug targeting HOX-high AML patient cells. The horizontal dashed line corresponds to p=0.05 and the vertical dashed line indicate a 2.5-fold difference in EC₅₀ values. FIG. 2C: AML patient samples included in the validation screen comprising HOX-high (dark grey, upper right) and HOX-med/low (light grey) specimens. FIG. 2D: Differential EC₅₀ values in HOX-high versus HOX-med/low AML samples measured in the validation screen. The p-value was determined by Mann-Whitney test. FIG. 2E: Frequencies of Mubritinib EC₅₀ values measured in 200 different AML specimens define Mubritinib-sensitive (EC₅₀<375 nM, n=100) and Mubritinib-resistant (EC₅₀≥375 nM, n=100) groups. Normal primitive CD34-positive cord blood cells were moderately sensitive to Mubritinib (EC₅₀>375 nM). FIG. 2F: Differential overall survival of patients belonging to the Mubritinib-sensitive versus-resistant groups. The p-value was determined by log-rank test. FIG. 2G: Transcriptomic profiles of Mubritinib-sensitive versus Mubritinib-resistant specimens, highlighting an overexpression of HOX-network genes. FIG. 2H: Transcriptomic profiles of Mubritinib-sensitive versus Mubritinib-resistant specimens, highlighting most differentially expressed genes (criteria: log (fold change)>0.8 (=6-fold), RPKM>0.1). FIG. 2I: List of under-expressed (above the dotted line) or over-expressed (below the dotted line) genes in Mubritinib-sensitive (n=100, separation by median of the entire cohort, EC₅₀=375 nM) versus Mubritinib-resistant (n=100) AML specimens according to false-discovery rate (FDR) corrected multiple Mann-Whitney test applied to RNA-sequencing data. Cut-offs used: expression >0.1RPKM, log(fold-change) between sensitive and resistant specimens >0.7 (=5-fold difference in gene expression). Abbreviations: AML: Acute myeloid leukemia; ANKRDI8B: Ankyrin Repeat Domain 18B; BEND6: BEN Domain Containing 6; COL4A5: Collagen Type IV Alpha 5; FDR: False Discovery Rate; HOX: Homeobox; KIRREL: Kin Of IRRE Like; LINC: Long Non Coding; LSC: leukemic stem cell; MEIS1: MEIS Homeobox 1; MIR: Micro RNA; MSLN: Mesothelin; NKX2.3: NK2 Homeobox 3; PI3K: Phosphatidylinositol-4,5-bisphosphate 3-kinase; PPBP: Pro-Platelet Basic Protein; PRDM16: PR/SET Domain 16; PRG3: Proteoglycan 3; RPKM: Reads Per Kilobase per Million mapped reads; RTK: receptor tyrosine kinase; S100A16: S100 Calcium Binding Protein A16; SNORD: Small Nucleolar RNA; ST18: Suppression Of Tumorigenicity 18, Zinc Finger; ZNF: Zinc Finger Protein.

FIGS. 3A-I show a characterization of Mubritinib-sensitive AML specimens according to various genetic and clinical features. FIG. 3A: Clinical features enriched in Mubritinib sensitive (EC₅₀<375 nM, n=100) versus resistant (EC₅₀≥375 nM, n=100) AML specimens according to a Bonferroni corrected exact Fisher's test. FIG. 3B: Mubritinib EC₅₀ values according to cytogenetic risk classes. FIG. 3C: Mutations enriched in highly-sensitive Mubritinib AML specimens (EC₅₀<100 nM, n=59) versus Mubritinib highly-resistant AML specimens (EC₅₀>1 μM, n=58) according to a Bonferroni corrected exact Fisher's test. FIG. 3D: Mubritinib EC₅₀ values according to the presence of mutated genes. FIG. 3E: Mubritinib EC₅₀ values according to the genetic subgroups. FIG. 3F: Summary of Mubritinib-sensitive patient sample characteristics. FIG. 3G: EC₅₀ values of poor prognostic patient specimens carrying mutations (m) in NPM1, FLT3-ITD and DNMT3A (Papaemmanuil, E. et al. N Engl J Med 374, 2209-2221) versus other AML samples. FIG. 3H-I: Leukemic stem cell (LSC) frequencies in Mubritinib sensitive versus resistant groups of patient samples belonging to the normal karyotype (NK) subgroup (FIG. 3H) and to the NK subgroup carrying mutated NPM1 (NPM1m) (FIG. 3I). In FIGS. 3E-I, p-values were calculated by Mann-Whitney test. Horizontal lines in all panels indicate median values. Abbreviations: AML: Acute myeloid leukemia; AbnChr: abnormal chromosome; ASXL1: Additional Sex Combs Like 1, Transcriptional Regulator; CEBPA: CCAAT/Enhancer Binding Protein Alpha; Complex: complex karyotype; DNMT3A: DNA (Cytosine-5-)-Methyltransferase 3 Alpha; EVI1: Ecotropic Viral Integration Site 1; FLT3: Fms Related Tyrosine Kinase 3; HOX: Homeobox; IDH: Isocitrate Dehydrogenase (NADP(+)); Inter(AbnK): intermediate cytogenetic risk with abnormal karyotype; m; mutated; MLL: Mixed Lineage Leukemia 1; NK: normal karyotype; NPM1: Nucleophosmin (Nucleolar Phosphoprotein B23, Numatrin); NRAS: Neuroblastoma RAS Viral Oncogene Homolog; NUP98: Nucleoporin 98 kDa; RUNX1: Runt Related Transcription Factor 1; SRSF2: Serine/Arginine-Rich Splicing Factor 2; TET2: Tet Methylcytosine Dioxygenase 2; TP53: Tumor Protein P53; WT1: Wilms Tumor 1; +8: trisomy 8.

FIGS. 4A-4G show the results on experiments assessing the effect of Mubritinib on tumor cells. FIG. 4A: Number of viable (Propidium idodide (PI)-negative) cells after four days of treatment of OCI-AML3 with different concentrations of Mubritinib. FIG. 4B: Fold-enrichment in PI-positive cells (dead cells) compared to DMSO-treated cells after 27 hours of treatment of OCI-AML3 with different concentrations of Mubritinib. FIG. 4C: Percentage of early apoptotic and late apoptotic cells in OCI-AML3 cells after 24 hours of treatment with control DMSO, 100 nM or 10 μM Mubritinib. FIG. 4D: AML patients' cell sensitivity to Mubritinib versus Lapatinib ditosylate, an ERBB2 inhibitor. FIG. 4E: OCI-AML3 dose response curves after treatment with Mubritinib or with two known ERBB2 inhibitors: Lapatinib ditosylate or Sapitinib. FIG. 4F: ERBB2 gene expression in Mubritinib-sensitive versus Mubritinib-resistant patient samples. FIG. 4G: ERBB2 protein expression versus isotype control in ERBB2 over-expressing BT474 breast cancer cell line and in OCI-AML3 Mubritinib-sensitive AML cell line measured by flow cytometry. Samples were either treated with Mubritinib at 2 μM for 24 hours or mock-treated with DMSO. Abbreviations: AML: Acute myeloid leukemia; ERBB2: Erb-B2 Receptor Tyrosine Kinase 2; FITC: Fluorescein isothiocyanate; PE: Phycoerythrin; Pos: positive.

FIGS. 5A-5J show the results on experiments assessing the mode of action of Mubritinib for mediating tumor cell death. FIG. 5A: OCI-AML3 leukemic cells were either pre-incubated for 4 hours with 6 mM N-acetyl-cysteine (NAC), a reactive oxygen species (ROS) scavenger, or with vehicle (water) before being treated with 100 nM Mubritinib for 24 h or vehicle (DMSO). Cells underwent apoptotic death upon Mubritinib treatment as assessed by Annexin V and propidium iodide (PI) staining by flow cytometry. Mubritinib-induced cell death was reduced when cells were cultured in the presence of NAC. FIG. 5B: Flow cytometric staining using 2′,7′-dichlorofluorescin diacetate (DCFDA), a fluorogenic dye that measures hydroxyl, peroxyl and other ROS activity within the cell, showing that Mubritinib treatment (500 nM, 24 h) induces ROS activity in OCI-AML3 leukemic cells. FIGS. 5C and 5D show the levels of reduced and oxidized levels of glutathione, respectively, in OCI-AML3 treated or not with Mubritinib, as detected by liquid chromatography-mass spectrometry (LC/MS). FIG. 5E shows the results of experiments assessing the effect of Mubritinib (1 μM) on oxygen consumption rate (OCR) in OCI-AML3 leukemic cells, as measured using a Seahorse XF® extracellular flux analyzer (Agilent®). FIG. 5F shows the results of experiments assessing the effect of Mubritinib on the mitochondrial electron transfer chain (ETC) complex I (pivotal for mitochondrial respiration/activity), as measured using a cell free assay (MitoTox™ Complex I OXPHOS activity microplate assay, Abcam). FIG. 5G is a graph depicting dose-response curves upon Mubritinib treatment (6-day culture assay in 384-well plates) of OCI-AML3 and OCI-AML5 cell lines. FIGS. 5H-J are graphs depicting dose-response curves following treatment of OCI-AML3 and OCI-AML5 cell lines with other ETC inhibitors, namely Oligomycin (inhibitor of complex V, FIG. 5H), Rotenone (inhibitor of complex I, FIG. 5I) and Deguelin (inhibitor of complex I, FIG. 5J).

FIGS. 6A-6E are graphs showing the effects of Mubritinib treatment on the levels of different intermediates of the citric acid cycle in OCI-AML3 cells, namely citrate (FIG. 6A), alpha-ketoglutarate (FIG. 6B), succinate (FIG. 6C), fumarate (FIG. 6D), and malate (FIG. 6E).

FIG. 7 is a graph showing the inhibitory effects of Mubritinib and Metformin hydrochloride on AML specimens. 20 AML specimens were tested for sensitivity to Mubritinib and Metformin hydrochloride at 1 μM in leukemic stem cell activity conservative culture conditions (Pabst et al., 2014, Nature Methods 11(4):436-42).

FIG. 8 is a graph showing the effect of Mubritinib on Complex I enzyme activity in OCI-AML3 cells, as assessed using the Complex I Enzyme Activity Microplate Assay Kit according to the manufacturer's instructions (Abcam, catalog No. ab109721). Complex I activity is determined by following the oxidation of NADH to NAD+ and the simultaneous reduction of a dye which leads to increased absorbance at OD=450 nm.

### DISCLOSURE OF INVENTION

Terms and symbols of genetics, molecular biology, biochemistry and nucleic acid used herein follow those of standard treatises and texts in the field, e.g. Green and Sambrook, Molecular Cloning: A Laboratory Manual, 4^{th} Edition, 2012 (Cold Spring Harbor Laboratory Press); Ausubel et al., Current Protocols in Molecular Biology (2001 and later updates thereto); Kornberg and Baker, DNA Replication, Second Edition (W University Science Books, 2005); Lehninger, Biochemistry, sixth Edition (WH Freeman & Co (Sd), New York, 2012); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like. All terms are to be understood with their typical meanings established in the relevant art.

The articles “a” and “an” are used herein to refer to one or to more than one (i.e. to at least one) of the grammatical object of the article. By way of example, “an element” means one element or more than one element. Throughout this specification, unless the context requires otherwise, the words “comprise,” “comprises” and “comprising” will be understood to imply the inclusion of a stated step or element or group of steps or elements but not the exclusion of any other step or element or group of steps or elements.

The information, including the nucleotide and amino acid sequences, corresponding to the Genbank, RefSeq, UniProt, NCBI and/or Ensembl accession numbers referred to in the present specification is incorporated herein by reference.

All methods described herein can be performed in any suitable order unless otherwise indicated herein or otherwise clearly contradicted by context.

The use of any and all examples, or exemplary language (“e.g.”, “such as”) provided herein, is intended merely to better illustrate the invention and does not pose a limitation on the scope of the invention unless otherwise claimed.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

Any and all combinations and subcombinations of the embodiments and features disclosed herein are encompassed by the present invention. For example, the expression of any combination of 2, 3, 4, 5 or more of the genes identified herein may be used in the methods described herein.

The terms “subject” and “patient” are used interchangeably herein, and refer to an animal, preferably a mammal, most preferably a human. In an embodiment, the patient is an adult AML patient. In an embodiment, the patient is less than 60 years old. In another embodiment, the patient is 60 years old or older. In another embodiment, the AML patient is a pediatric AML patient.

The term “effective amount” as used herein refers to that amount of the active agent being administered (a mitochondrial activity inhibitor, for example Mubritinib or a pharmaceutically acceptable salt thereof) which will relieve to some extent one or more of the symptoms of the disease (AML) being treated (AML), for example that will inhibit the growth and/or induce cell death in the AML cells. The effective amount will be adjusted to minimize adverse effects, for example the inhibition of the growth and/or induction of cell death in normal cells.

In the studies described herein, it is shown that AML cells expressing high levels of HOX-network genes, which are associated with poor prognosis/survival, are sensitive to Mubritinib. Mubritinib-sensitive AML cells were also shown to be enriched in AML samples having certain features, such as for example overexpression or underexpression of specific genes, certain cytogenetic or molecular risk factors, such as Normal Karyotype (NK), mutated NPM1, FLT3-ITD and DNMT3A specimens, and in specimens having high leukemic stem cell (LSC) frequencies. It is also demonstrated that Mubritinib, characterized as an inhibitor of the tyrosine kinase human epidermal growth factor receptor 2 (HER2/ErbB2), does not target this protein in AML cells, and compelling evidence that Mubritinib induces AML cell apoptosis through inhibition of mitochondrial activity/respiration, more particularly of the electron transport chain (ETC), resulting in increased ROS production in sensitive AML cells, are provided. Other inhibitors of mitochondrial activity/respiration were also shown to induce apoptosis in Mubritinib-sensitive AML cells.

Accordingly, in an aspect, the present disclosure provides a method for treating AML, for example poor prognosis or poor risk AML, said method comprising administering to a subject in need thereof an effective amount of a mitochondrial activity inhibitor, e.g., an electron transport chain (ETC) inhibitor. The present disclosure also provides the use of a mitochondrial activity inhibitor, e.g., an ETC inhibitor, for treating a subject suffering from AML, for example poor prognosis or poor risk AML, or for the manufacture of a medicament for treating a subject suffering from AML, for example poor prognosis or poor risk AML. The present disclosure also provides a mitochondrial activity inhibitor, e.g., an ETC inhibitor, for use in the treatment of a subject suffering from AML, for example poor prognosis or poor risk AML.

In another aspect, the present disclosure provides a method for determining whether an agent may be suitable for treating AML, for example poor prognosis or poor risk AML, said method comprising determining whether said agent inhibits mitochondrial activity or respiration (e.g., inhibits the ETC) in a biological system (e.g. a cell or a cell extract), wherein inhibition of mitochondrial activity is indicative that the agent may be suitable for treating AML.

The term “mitochondrial activity inhibitor” (or “mitochondrial respiration inhibitor”) as used herein refers to an inhibitor of the oxidative cellular energy production process, typically an inhibitor of the aerobic cell metabolism. An “oxidative cellular energy production process inhibitor” includes an inhibitor of the cellular tricarboxylic acid (TCA) cycle (also known as the citric acid cycle, CAC, or Krebs cycle) (chemical conversion of carbohydrates, fats and proteins into carbon dioxide and water to generate a form of usable energy), or an inhibitor of the cellular oxidative (aerobic) glycolysis (metabolism of glucose to pyruvate in the cell cytoplasm) or of the oxidative phosphorylation of glycolysis substrate (pyruvate). The mitochondrial activity inhibitor according to the present disclosure is an agent which exhibits a capacity to block the ETC or oxidative phosphorylation, leading to the production of Reactive Oxygen Species (ROS, such as H₂O₂) (i.e. increase in the levels of ROS in the cells), i.e. is an ROS-inducing mitochondrial activity inhibitor. The effective amount of the mitochondrial activity inhibitor is an amount that is toxic to AML cells (inhibit AML cell proliferation and/or induce AML cell death) but not toxic (or significantly less toxic) for normal, non-AML, cells.

In an embodiment, the mitochondrial activity inhibitor is a TCA cycle inhibitor, for example an inhibitor of one or more of pyruvate dehydrogenase, citrate synthase, aconitase, isocitrate lyase, alpha-ketoglutarate dehydrogenase complex, succinyl CoA synthetase, succinate dehydrogenase, fumarase, malate synthase, glutaminase and pyruvate dehydrogenase complex. Several inhibitors of the TCA cycle are known in the art. Examples of TCA cycle inhibitors include depletors of NAD+ and/or NADH (e.g., hypoglycin A and its metabolite methylenecyclopropylacetic acid, ketone bodies such as D(−)-3-hydroxybutyrate, alloxan, PNU), inhibitors of pyruvate dehydrogenase such as arsenite, dichlorovinyl-cysteine, p-benzoquinone, and thiaminase, inhibitors of citrate synthetase such as fluoroacetate, halogenated acetates (iodo-, bromo-, chloro-acetate), fluoroacetamide, halogenated acetyl-CoA (fluoroacetyl-CoA, bromoacetyl-CoA, chloroacetyl-CoA, iodoacetyl-CoA), halogenated crotonate (fluoro-, iodo-, bromo-, chloro-crotonate), halogenated ketone bodies, (chloro-, fluoro-, bromo-, iodoaceto-acetate, fluoro-, chloro-, bromo-, iodo-butyrate, fluoro-, chloro-, bromo-, iodo-acetone), and halogenated oleate (iodo-, bromo-, chloro-, fluoro-oleate), inhibitors of aconitase such as halogenated citrate, and halogenated citrate 2R,3R isomer (fluoro-, bromo-, chloro-, iodo-citrate), inhibitors of isocitrate dehydrogenase such as dichlorovinyl-cysteine (DCVC), inhibitors of succinate dehydrogenase such as malonate, DCVC, pentachlorobutadienyl-cysteine, 2-bromohydroquinone, 3-nitropropionic acid, and cis-crotonalide fungicides, inhibitors of glutaminase such as 6-diazo-5-oxo-L-norleucine (DON), other TCA cycle inhibitors such as glu-hydroxyoxamate, p-chloromercuriphenylsulphonic acid, L-glutamate gamma-hydroxamate, p-chloromercuriphenylsulphonic acid, acivicin (alpha-amino-3-chloro-4,5-dihydro-5-isoxazoleacetic acid, halogenated glutamine (fluoro-, iodo-, chloro-, bromo-glutamine), or halogenated glutamate (fluoro-, iodo-, chloro-, bromo-glutamate), halogenated amino acids, as well as analogs, derivatives, and salts thereof.

The ETC (also known as the respiratory chain) is a series of protein complexes located in the intermembrane space of the mitochondria of eukaryotic cells that transfer electrons from electron donors to electron acceptors via redox reactions, and couples this electron transfer with the transfer of protons (H⁺ ions) across a membrane, which creates an electrochemical proton gradient that drives the synthesis of adenosine triphosphate (ATP). The components of the ETC are organized into 4 complexes (Complexes I to IV), and each complex contains several different electron carriers. Complex I, also known as the NADH-coenzyme Q reductase or NADH dehydrogenase) accepts electrons from NADH and serves as the link between glycolysis, the citric acid cycle, fatty acid oxidation and the ETC. Complex II, also known as succinate-coenzyme Q reductase or succinate dehydrogenase, includes succinate dehydrogenase and serves as a direct link between the citric acid cycle and the ETC. Complexes I and II both produce reduced coenzyme Q, CoQH₂ which is the substrate for Complex III. Complex III, also known as coenzyme Q reductase, transfers the electrons from CoQH₂ to reduce cytochrome c which is the substrate for Complex IV. Finally, Complex IV, also known as cytochrome c reductase, transfers the electrons from cytochrome c to reduce molecular oxygen into water.

The term “ETC inhibitor” as used herein refers an inhibitor of any stage of mitochondrial electron transport, e.g., an inhibitor of Complex I, Complex II, Complex III and/or Complex IV of the human mitochondrial ETC, and which induces the production of ROS in cells (i.e. increase the levels of ROS relative to an untreated cells), i.e. is an ROS-inducing ETC inhibitor.

Several inhibitors of the ETC are known in the art. Examples of ETC inhibitors include rotenone and its derivatives (e.g., deguelin, arylazidoamorphigenin, amorphispironone, tephrosin, amorphigenin, 12a-hydroxyamorphigenin, 12a-hydroxydalpanol and 6′-O-D-glucopyranosyldalpanol), thenoyltrifluoroacetone (TTFA), diphenyleneiodonium chloride (DPI), phenformin, Rolliniastatin 1 and 2, amytal, 2-heptyl-4-hydroxyquinoline, antimycin A1 and its derivatives (e.g., myxothiazol), organochalcogens such as ebselen (Ebs), diphenyl diselenide (PhSe)₂ and diphenyl ditelluride (PhTe)₂, dual serotonin-noradrenaline re-uptake inhibitors (e.g., venlafaxine, O-desmethylvenlafaxine, clomipramine, desmethylclomipramine, imipramine, duloxetine, milnacipran and chlorpromazine), Licochalcone A, Ascochlorin, Strobilirubin B, Piericidin, imipramine NADH-linked enzyme inhibitors such as 2-anthracene-carboxylic acid, NADH-linked enzyme antagonists such as a-NADH, and copper-chelating agent such as diethyldithiocarbamate. Inhibitors of the activity of complex III of the ETC are described in PCT publications Nos. WO2012/0700015 (e.g., N-cyclononyl-3-formamido-2-hydroxybenzamide) and WO2013/174947 (e.g., 3-formamido-N-(heptadecan-9-yl)-2-hydroxybenzamide, N-cyclopentadecyl-7-hydroxy-1H-indazole-6-carboxamide, 3,5-dichloro-N-(3-(3-chloro-10,11-dihydro-5H-dibenzo[b,f]azepin-5-yl)-2-hydroxybenzamide, 2-(3-formamido-2-hydroxybenzamido)-2,3-dihydro-1H-inden-1-yl octanoate, N-cyclopentadecyl-3-formamido-2-hydroxybenzamide, N-cyclododecyl-3-formamido-2-hydroxybenzamide, 3,5-dichloro-N-cyclopentadecyl-2-hydroxybenzamide, 3,5-dichloro-N-decyl-2-hydroxybenzamide, N-(3,5-dichloro-2-hydroxyphenyl)undecanamide, N-(4-(cyclopentadecylcarbamoyl)phenyl)-3-formamido-2-hydroxybenzamide, N-(heptadecan-9-yl)-2-oxo-2,3-dihydro-1H-benzo[d]imidazole-4-carboxamide, N-(3-(3-chloro-10,11-dihydro-5H-dibenzo[b,t]azepin-5-yn)propyl)-7-hydroxy-1H-imidazole-6-carboxamide, N-(3-(3-chloro-10,11-dihydro-5H-dibenzo[b,f]azepin-5-yl)propyl)-3-fomamido-2-hydroxybenzamide, N-(3-(10,11-dihydro-5H-dibenzo[b,f]azepin-5-yl)propyl)-3-fomamido-2-hydroxybenzamide, N-(3-(1-(heptadecan-9-yl)-1H-1,2,3-triazol-4-yl)-2-hydroxyphenyl)formamide, N-(3-(1-cyclopentadecyl-1H-1,2,3-triazol-4-yl)-2-hydroxyphenyl)formamide, N-cyclononyl-7-hydroxy-1H-indazole-6-carboxamide, N-cyclononyl-2-oxo-2,3-dihydro-1H-benzo[d]imidazole-4-carboxamide, and N-cyclopentadecyl-2-oxo-2,3-dihydro-1H-benzo[d]imidazole-4-carboxamide), as well as analogs, derivatives, and salts thereof.

In an embodiment, the ETC inhibitor inhibits the activity of the Complex I and/or Complex III of the human mitochondrial ETC, which are considered the major sites for ROS production. In an embodiment, the ETC inhibitor blocks the activity of the Complex III of the human mitochondrial ETC. In another embodiment, the ETC inhibitor blocks the activity of the Complex (I) of the human mitochondrial ETC. The complex I inhibitor is a class A complex I inhibitor according to the classification of Fato et al. (Biochim Biophys Acta, 2009 May; 1787(5): 384-392). The term “class A complex I inhibitor” as used herein refers to an inhibitor of complex I that induces the production of ROS in cells, i.e. is an ROS-inducing complex I inhibitor. Examples of class A complex I inhibitors include rotenone, piericidin A and rolliniastatin 1 and 2 In an embodiment, the class A complex I inhibitor binds to, or near, the quinone-binding site of complex I.

As noted above, the studies described herein have provided evidence that Mubritinib induces AML cell apoptosis through inhibition of mitochondrial activity, more particularly of the ETC, resulting in induction of ROS production in the leukemic cells. Mubritinib exhibits a mechanism of action corresponding to class A complex I inhibitors.

Thus, in an embodiment, the ETC inhibitor is Mubritinib or a pharmaceutically acceptable salt thereof. Thus, in another aspect, the present disclosure relates to the use of Mubritinib or a pharmaceutically acceptable salt thereof for inhibiting mitochondrial activity or respiration, e.g., for inhibiting the ETC, in a cell.

In another aspect, the present disclosure provides a method for treating AML, for example poor prognosis or poor risk AML, said method comprising administering to a subject in need thereof an effective amount of Mubritinib or a pharmaceutically acceptable salt thereof. The present disclosure also provides the use of Mubritinib or a pharmaceutically acceptable salt thereof, for treating a subject suffering from AML, for example poor progniosis or poor risk AML, or for the manufacture of a medicament for treating a subject suffering from AML, for example poor prognosis or poor risk AML. The present disclosure also provides Mubritinib or a pharmaceutically acceptable salt thereof, for use in the treatment of a subject suffering from AML, for example poor prognosis or poor risk AML.

In another aspect, the present disclosure provides a method for treating poor prognosis or poor risk AML, said method comprising administering to a subject in need thereof an effective amount of Mubritinib or a pharmaceutically acceptable salt thereof.

Mubritinib (1-(4-{4-[(2-{(E)-2-[4-(trifluoromethyl)phenyl]ethenyl}-1,3-oxazol-4-yl)methoxy]phenyl}butyl)-1H-1,2,3-triazole, CAS Number 366017-09-6), also referred to as TAK-165, has the following structure:

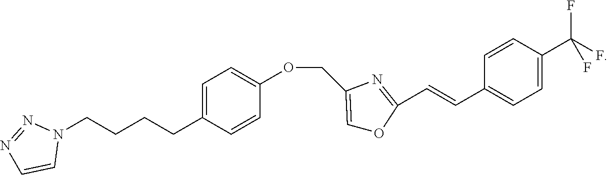 [SMILES: FC(F)(F)c1ccc(/C=C/c2nc(COc3ccc(CCCCn4ccnn4)cc3)co2)cc1]

It is described as a potent inhibitor of human epidermal growth factor receptor 2 (ERBB2HER2) (Nagasawa et al., Int J Urol. 2006 May; 13(5):587-92). Methods to synthesize Mubritinib are known in the art, and are described, for example, in PCT publication No. WO/2001/77107. In an embodiment, the methods and used described herein comprises the use or administration of Mubritinib.

The present inventors have shown that AML specimens more sensitive to Mubritinib have certain characteristics/features, including (i) higher expression of certain genes (see Table 1), notably homeobox (HOX)-network genes, relative to AML specimens more resistant to Mubritinib, (ii) lower expression of certain genes (see Table 2), relative to AML specimens more resistant to Mubritinib; (iii) certain cytogenetic or molecular risk factors, such as intermediate cytogenetic risk, Normal Karyotype (NK), high HOX status, mutated NPM1, mutated CEBPA, mutated FLT3, mutated DNA methylation genes (DNMT3A, TET2, IDH1, IDH2), mutated RUNX1, mutated WT1, mutated SRSF2, intermediate cytogenetic risk with abnormal karyotype (intern(abnK)), trisomy 8 (+8) and abnormal chr(5/7); and (iv) a higher leuikemic stem cell (LSC) frequency, i.e. an LSC frequency of about 1 LSC per 1×10⁶ total cells or more, relative to AML specimens more resistant.

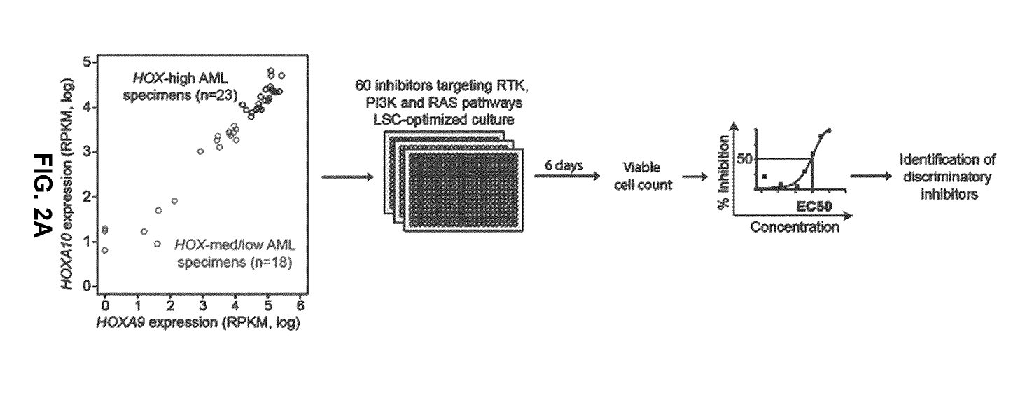

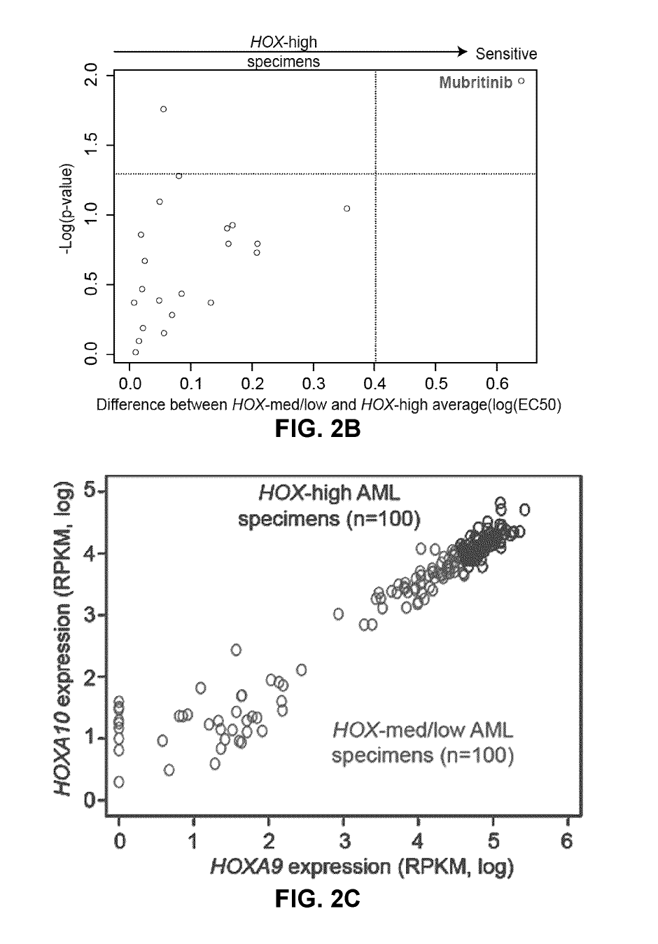

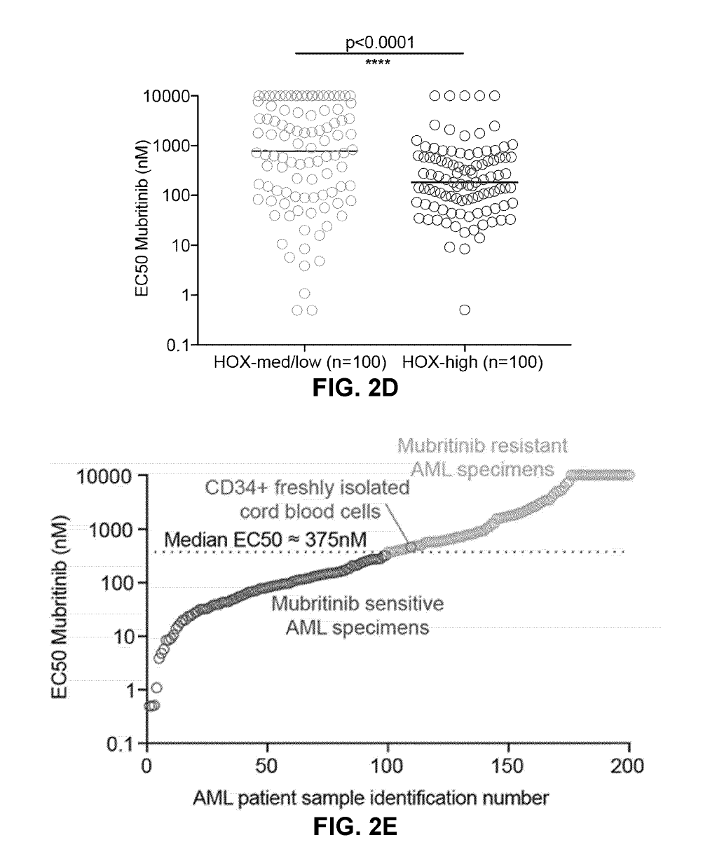

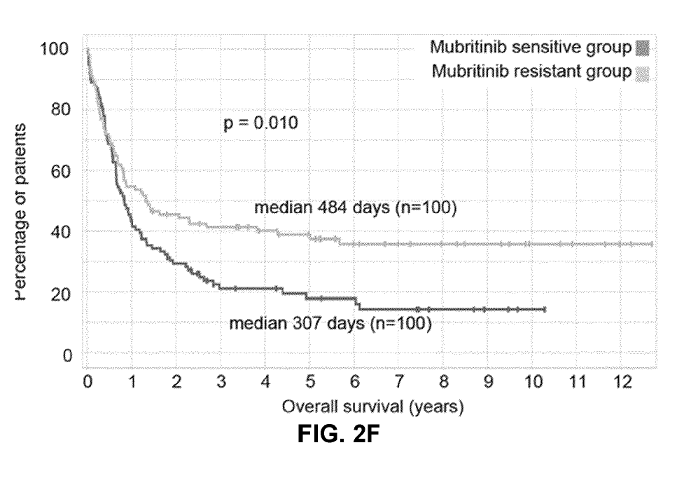

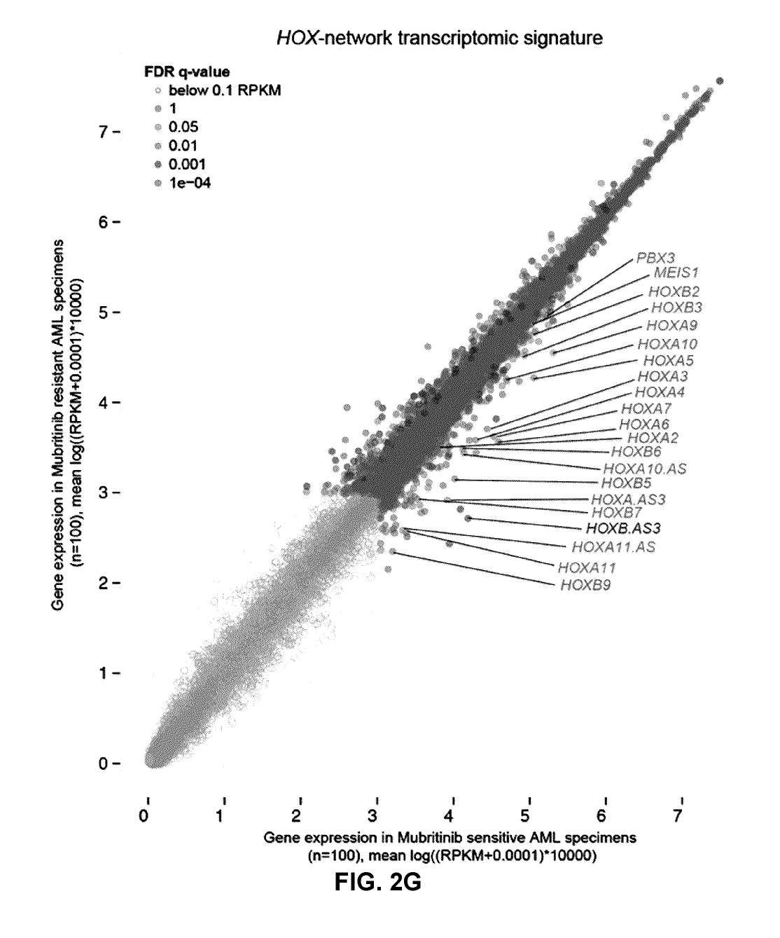

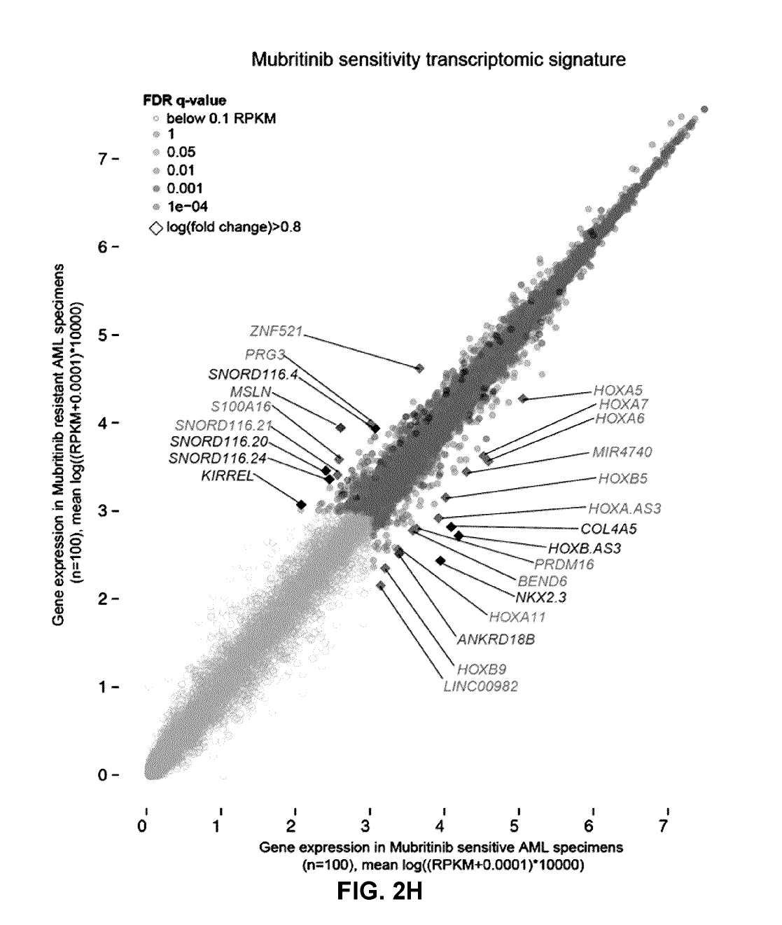

TABLE 1
Genes overexpressed in Mubritinib sensitive
versus resistant AML specimens (see FIG. 2I)

| | | | | | |
|---|---|---|---|---|---|
| HOXA5 | HOXA9 | PRDM16 | LOC285758 | HOXA3 | HOXA.AS3 |
| HOXB5 | HOXA11 | BEND6 | MIR4740 | COL4A5 | HOXA6 |
| HOXB9 | HOXA10.AS | LINC00982 | CYP7B1 | ANKRD18B | |
| HOXA4 | HOXA11.AS | NKX2.3 | HOXA7 | HOXB.AS3 | |

TABLE 2
Genes underexpressed in Mubritinib sensitive
versus resistant AML specimens (see FIG. 2I)

| | | | | |
|---|---|---|---|---|
| ORM1 | SNORD116.4 | MSLN | MS4A2 | PRG3 |
| PRAME | SNORD116.24 | TINAGL1 | SNORD116.20 | ST18 |
| MYZAP | ZNF521 | S100A16 | KIRREL | SNORD116.21 |

Thus, in an embodiment, the subject treated by the method/use described herein suffers from AML characterized by at least one of the following features: (a) high level of expression of one or more homeobox (HOX)-network genes; (b) high level of expression of one or more of the genes depicted in Table 1; (c) low level of expression of one or more of the genes depicted in Table 2; (d) one or more of the following cytogenetic or molecular risk factor: intermediate cytogenetic risk, Normal Karyotype (NK), mutated NPM1, mutated CEBPA, mutated FLT3, mutated DNA methylation genes, mutated RUNX1, mutated WT1, mutated SRSF2, intermediate cytogenetic risk with abnormal karyotype (intern(abnK)), trisomy 8 (+8) and abnormal chr(5/7); and (e) a leukemic stem cell (LSC) frequency of about 1 LSC per 1×10⁶ total cells, or more.

In another aspect, the present disclosure provides a method for treating AML, said method comprising administering to a subject in need thereof an effective amount of a mitochondrial activity inhibitor, for example Mubritinib or a pharmaceutically acceptable salt thereof, wherein said AML has at least one of the following features: (a) high level of expression of one or more HOX-network genes; (b) high level of expression of one or more of the genes depicted in Table 1; (c) low level of expression of one or more of the genes depicted in Table 2; (d) one or more of the following cytogenetic or molecular risk factor: intermediate cytogenetic risk, Normal Karyotype (NK), mutated NPM1, mutated CEBPA, mutated FLT3, mutated DNA methylation genes, mutated RUNX1, mutated WT1, mutated SRSF2, intermediate cytogenetic risk with abnormal karyotype (intern(abnK)), trisomy 8 (+8) and abnormal chr(5/7); and (e) a leukemic stem cell (LSC) frequency of about 1 LSC per 1×10⁶ total cells, or more.

In another aspect, the present disclosure provides a use of a mitochondrial activity inhibitor, for example Mubritinib or a pharmaceutically acceptable salt thereof, for treating a subject suffering from AML, wherein said AML has at least one of the features (a)-(e) defined above. In another aspect, the present disclosure provides a use of a mitochondrial activity inhibitor, for example Mubritinib or a pharmaceutically acceptable salt thereof, for the manufacture of a medicament for treating a subject suffering from AML, wherein said AML has at least one of the features (a)-(e) defined above. In another aspect, the present disclosure provides a mitochondrial activity inhibitor, for example Mubritinib or a pharmaceutically acceptable salt thereof, for use in the treatment of a subject suffering from AML, wherein said AML has at least one of the features (a)-(e) defined above.

In an embodiment, the subject to be treated has already been identified has having one or more of the above-noted features (a)-(e). In another embodiment, the methods further comprise a step of identifying a subject having one or more of the above-noted features, e.g. by performing a suitable assay, prior to administration of the mitochondrial activity inhibitor, for example Mubritinib or pharmaceutically acceptable salt thereof.

In another aspect, the present disclosure provides a method for determining a suitable therapy for a subject suffering from AML, the method comprising determining the presence or absence of at least one of features (a)-(e) defined herein in an AML cell sample from the subject, wherein the presence of at least one of features (a)-(e) is indicative that the subject is a suitable candidate for a therapy comprising a mitochondrial activity inhibitor, for example Mubritinib or a pharmaceutically acceptable salt thereof, and wherein the absence of features (a)-(e) is indicative that the subject is not a suitable candidate for a therapy comprising mitochondrial activity inhibitor, for example Mubritinib or a pharmaceutically acceptable salt thereof, i.e. is a candidate for a therapy free of a mitochondrial activity inhibitor.

In another aspect, the present disclosure provides a method for determining a suitable therapy for a subject suffering from AML, the method comprising determining the presence or absence of at least one of features (a)-(e) defined herein in an AML cell sample from the subject, wherein the presence of at least one of features (a)-(e) is indicative that the subject is not a suitable candidate for a therapy comprising a mammalian target of rapamycin (mTOR) kinase inhibitor (i.e. is a candidate for a therapy free of mTOR kinase inhibitor), and wherein the absence of features (a)-(e) is indicative that the subject is a suitable candidate for a therapy comprising a mammalian target of rapamycin (mTOR) kinase inhibitor, e.g., AZD-2014 (3-(2,4-bis((S)-3-methylmorpholino)pyrido[2,3-d]pyrimidin-7-yl)-N-methylbenzamide, Vistusertib), AZD-8055 ((5-(2,4-bis((S)-3-methylmorpholino)pyrido[2,3-d]pyrimidin-7-yl)-2-methoxyphenyl)methanol), OSI-027 ((1r,4r)-4-(4-amino-5-(7-methoxy-1H-indol-2-yl)imidazo[1,5-f][1,2,4]triazin-7-yl)cyclohexanecarboxylic acid). In an embodiment, the method comprises determining the presence or absence of feature (a), i.e. high expression of one or more HOX-network genes, for example one or more of the HOX genes listed in Table 1, such as HOXA9 and/or HOXA10.

In another aspect, the present disclosure provides a method for treating poor prognosis or poor-risk AML, said method comprising administering to a subject in need thereof an effective amount of a mitochondrial activity inhibitor, preferably a class A complex I ETC inhibitor, for example Mubritinib or a pharmaceutically acceptable salt thereof. In another aspect, the present disclosure provides a use of a mitochondrial activity inhibitor, preferably a class A complex I ETC inhibitor, for example Mubritinib or a pharmaceutically acceptable salt thereof, for treating poor prognosis or poor-risk AML in a subject. In another aspect, the present disclosure provides a use of a mitochondrial activity inhibitor, preferably a class A complex I ETC inhibitor, for example Mubritinib or a pharmaceutically acceptable salt thereof, for the manufacture of a medicament for treating poor prognosis or poor-risk AML in a subject. In another aspect, the present disclosure provides a mitochondrial activity inhibitor, preferably a class A complex I ETC inhibitor, for example Mubritinib or a pharmaceutically acceptable salt thereof, for use in the treatment of poor prognosis or poor-risk AML in a subject.

In another aspect, the present disclosure provides a method for treating intermediate-risk AML, said method comprising administering to a subject in need thereof an effective amount of a mitochondrial activity inhibitor, preferably a class A complex I ETC inhibitor, for example Mubritinib or a pharmaceutically acceptable salt thereof. In another aspect, the present disclosure provides a use of a mitochondrial activity inhibitor, preferably a class A complex I ETC inhibitor, for example Mubritinib or a pharmaceutically acceptable salt thereof, for treating intermediate-risk AML in a subject. In another aspect, the present disclosure provides a use of a mitochondrial activity inhibitor, preferably a class A complex I ETC inhibitor, for example Mubritinib or a pharmaceutically acceptable salt thereof, for the manufacture of a medicament for treating intermediate-risk AML in a subject. In another aspect, the present disclosure provides a mitochondrial activity inhibitor, preferably a class A complex I ETC inhibitor, for example Mubritinib or a pharmaceutically acceptable salt thereof, for use in the treatment of intermediate-risk AML in a subject.

In another aspect, the present disclosure provides a method for treating poor- and/or intermediate-risk AML, said method comprising administering to a subject in need thereof an effective amount of a mitochondrial activity inhibitor, preferably a class A complex I ETC inhibitor, for example Mubritinib or a pharmaceutically acceptable salt thereof. In another aspect, the present disclosure provides a use of a mitochondrial activity inhibitor, preferably a class A complex I ETC inhibitor, for example Mubritinib or a pharmaceutically acceptable salt thereof, for treating poor- and/or intermediate-risk AML in a subject. In another aspect, the present disclosure provides a use of a mitochondrial activity inhibitor, preferably a class A complex I ETC inhibitor, for example Mubritinib or a pharmaceutically acceptable salt thereof, for the manufacture of a medicament for treating poor- and/or intermediate-risk AML in a subject. In another aspect, the present disclosure provides a mitochondrial activity inhibitor, preferably a class A complex I ETC inhibitor, for example Mubritinib or a pharmaceutically acceptable salt thereof, for use in the treatment of poor- and/or intermediate-risk AML in a subject.

As used herein, the term “prognosis” refers to the forecast of the probable outcome or course of AML; the patient's chance of recovery or survival. The terms “poor prognosis AML” or “poor-risk AML” as used herein refer to an AML associated with long-term survival (5 years or more) of less than about 25% or 20% based on the currently available therapies. Poor prognosis AML is often associated with, for example, deletion of part of chromosome 5 or 7, translocation between chromosomes 9 and 11, translocation or inversion of chromosome 3, translocation between chromosomes 6 and 9, translocation between chromosomes 9 and 22, abnormalities of chromosome 11, FLT3 gene mutations, high EVI1 expression, complex karyotype (>3 abnormalities), and high HOX-network genes expression.

The term “intermediate-risk AML” as used herein refers to an AML is associated with long-term survival (5 years or more) of between about 60% to about 25% based on the currently available therapies. intermediate-risk AMLs are generally not associated with favorable and particular unfavorable cytogenetic aberrations (i.e. “uninformative” cytogenetic aberrations), and account for a significant proportion (approximately 55%) of AML patients. Examples of intermediate-risk AMLs include normal karyotype (NK) AML, NUP98-NSD1 fusion in AML with normal karyotype (NK), trisomy 8 alone AML, and intermediate abnormal karyotype AML.

In another aspect, the present disclosure provides a method for treating AML (e.g., poor-risk or poor prognosis AML), said method comprising administering to a subject in need thereof an effective amount of a mitochondrial activity inhibitor, preferably a class A complex I ETC inhibitor, for example Mubritinib or a pharmaceutically acceptable salt thereof, wherein said AML exhibiting high level of expression of one or more HOX-network genes (i.e. HOX-high AML). In another aspect, the present disclosure provides a use of a mitochondrial activity inhibitor, preferably a class A complex I ETC inhibitor, for example Mubritinib or a pharmaceutically acceptable salt thereof, for treating AML (e.g., poor-risk or poor prognosis AML), said AML exhibiting high level of expression of one or more HOX-network genes. In another aspect, the present disclosure provides a use of a mitochondrial activity inhibitor, preferably a class A complex I ETC inhibitor, for example Mubritinib or a pharmaceutically acceptable salt thereof, for the manufacture of a medicament for treating AML (e.g., poor-risk or poor prognosis AML), said AML exhibiting high level of expression of one or more HOX-network genes. In another aspect, the present disclosure provides a mitochondrial activity inhibitor, preferably a class A complex I ETC inhibitor, for example Mubritinib or a pharmaceutically acceptable salt thereof, for use in the treatment of AML (e.g., poor-risk or poor prognosis AML), said AML exhibiting high level of expression of one or more HOX-network genes.

The present disclosure also provides a method for determining the likelihood that a subject suffering from AML responds to a treatment with a mitochondrial activity inhibitor, preferably a class A complex I ETC inhibitor, for example Mubritinib or a pharmaceutically acceptable salt thereof, the method comprising determining whether AML cells from said subject exhibit high level of expression of one or more HOX-network genes, wherein high level of expression of the one or more HOX-network genes in said AML cells is indicative that the subject has a high likelihood of responding to said treatment.

Deregulation of the HOX-MEIS-PBX network (HOX network) is a common molecular anomaly in AML patients (Lawrence, H. J. et al. Leukemia 13, 1993-1999 (1999)). It is detected for example in two AML subtypes: in AML patients with normal karyotype (NK) carrying a mutation in nucleophosmin gene (NK NPM1m, representing around 25% of patients) and in a subgroup of patients with chromosome translocations involving the mixed lineage leukemia gene (MLL) on 11q23 (8% of patients).

The term “HOX-network gene” as used herein refers to a gene involved in the regulatory network of transcription factor (TF) family HOX and expressed in cells of the hematopoietic lineage. Members of the “HOX-network gene” expressed in cells of the hematopoietic lineage includes HOXgenes of clusters A, B and C, such as HOXB1, HOXB2, HOXB3, HOXB4, HOXB5, HOXB6, HOXB7, HOXB9, HOXB-AS3, HOXA1, HOXA2, HOXA3, HOXA4, HOXA5, HOXA6, HOXA7, HOXA9, HOXA10, HOXA10-AS, HOXA11, HOXA11-AS and HOXA-AS3, as well as other genes such as MEIS1 and PBX3. In an embodiment, at least one HOX-network gene is highly expressed in the AML. In an embodiment, at least 2 HOX-network genes are highly expressed in the AML. In an embodiment, at least 3 HOX-network genes are highly expressed in the AML. In an embodiment, at least 4 HOX-network genes are highly expressed in the AML. In an embodiment, at least 5 HOX-network genes are highly expressed in the AML. In an embodiment, at least 10 HOX-network genes are highly expressed in the AML. In an embodiment, at least 15 HOX-network genes are highly expressed in the AML. In an embodiment, at least 20 HOX-network genes are highly expressed in the AML. In an embodiment, at least one of HOXA9 and HOXA10 are highly expressed in the AML. In an embodiment, both HOXA9 and HOXA10 are highly expressed in the AML. AML with high HOX gene expression defines a distinct biologic subset of AML, characterized by poor prognosis (adverse survival), intermediate risk cytogenetics, higher levels of FLT3 expression, frequent FLT3 and NPM1 mutations, and high LSC frequencies (see, e.g., Roche et al., Leukemia (2004) 18, 1059-1063; Kramarzova et al., Journal of Hematology & Oncology 2014 7:94). In an embodiment, the HOX-network gene(s) highly expressed in the AML is one or more of the HOX-network genes listed in Table 1.

In an embodiment, the above-mentioned method further comprises measuring the level of expression of one or more HOX-network genes in a sample comprising leukemic cells from the subject, and comparing the level to a reference level or control to determine whether the one or more HOX-network genes is/are highly expressed or overexpressed in the leukemic cells, and wherein if the one or more HOX-network genes is/are highly expressed or overexpressed in the leukemic cells, selecting the subject for treatment with a mitochondrial activity inhibitor, preferably a class A complex I ETC inhibitor, for example Mubritinib or a pharmaceutically acceptable salt thereof.

The present disclosure also provides a method for treating AML, said method comprising administering to a subject in need thereof an effective amount of a mitochondrial activity inhibitor, preferably a class A complex I ETC inhibitor, for example Mubritinib or a pharmaceutically acceptable salt thereof, wherein said AML exhibits high level of expression of one or more of the genes depicted in Table 1. In another aspect, the present disclosure provides a use of a mitochondrial activity inhibitor, preferably a class A complex I ETC inhibitor, for example Mubritinib or a pharmaceutically acceptable salt thereof, for treating a subject suffering from AML, wherein said AML exhibits high level of expression of one or more of the genes depicted in Table 1. In another aspect, the present disclosure provides a use of a mitochondrial activity inhibitor, preferably a class A complex I ETC inhibitor, for example Mubritinib or a pharmaceutically acceptable salt thereof, for the manufacture of a medicament for treating a subject suffering from AML, wherein said AML exhibits high level of expression of one or more of the genes depicted in Table 1. In another aspect, the present disclosure provides a mitochondrial activity inhibitor, preferably a class A complex I ETC inhibitor, for example Mubritinib or a pharmaceutically acceptable salt thereof, for use in the treatment of a subject suffering from AML, wherein said AML exhibits high level of expression of one or more of the genes depicted in Table 1.

The present disclosure also provides a method for determining the likelihood that a subject suffering from AML responds to a treatment with a mitochondrial activity inhibitor, preferably a class A complex I ETC inhibitor, for example Mubritinib or a pharmaceutically acceptable salt thereof, the method comprising determining whether AML cells from said subject exhibit high level of expression of one or more of the genes depicted in Table 1, wherein high level of expression of the one or more genes in said AML cells is indicative that the subject has a high likelihood of responding to said treatment.

In an embodiment, at least one gene of Table 1 is highly expressed (overexpressed) in the AML. In an embodiment, at least 2 genes of Table 1 are highly expressed in the AML. In an embodiment, at least 3 genes of Table 1 are highly expressed in the AML. In an embodiment, at least 4 genes of Table 1 are highly expressed in the AML. In an embodiment, at least 5 genes of Table 1 are highly expressed in the AML. In an embodiment, at least 10 genes of Table 1 are highly expressed in the AML. In an embodiment, at least 1, 2, 3, 4, 5, 10, or more of the genes listed in Table 1 are highly expressed in the AML. In an embodiment, all the genes listed in Table 1 are highly expressed in the AML.

In an embodiment, the above-mentioned method further comprises measuring the level of expression of one or more of the genes of Table 1 in a sample comprising leukemic cells from the subject, and comparing the level to a reference level or control to determine whether the one or more genes is/are highly expressed or overexpressed in the leukemic cells, and wherein if the one or more genes is/are highly expressed or overexpressed in the leukemic cells, selecting the subject for treatment with a mitochondrial activity inhibitor, preferably a class A complex I ETC inhibitor, for example Mubritinib or a pharmaceutically acceptable salt thereof.

The present disclosure also provides a method for treating AML, said method comprising administering to a subject in need thereof an effective amount of a mitochondrial activity inhibitor, preferably a class A complex I ETC inhibitor, for example for example Mubritinib or a pharmaceutically acceptable salt thereof, wherein said AML exhibits low level of expression of one or more of the genes depicted in Table 2. In another aspect, the present disclosure provides a use of a mitochondrial activity inhibitor, preferably a class A complex I ETC inhibitor, for example Mubritinib or a pharmaceutically acceptable salt thereof, for treating a subject suffering from AML, wherein said AML exhibits low level of expression of one or more of the genes depicted in Table 2. In another aspect, the present disclosure provides a use of a mitochondrial activity inhibitor, preferably a class A complex I ETC inhibitor, for example Mubritinib or a pharmaceutically acceptable salt thereof, for the manufacture of a medicament for treating a subject suffering from AML, wherein said AML exhibits low level of expression of one or more of the genes depicted in Table 2. In another aspect, the present disclosure provides a mitochondrial activity inhibitor, preferably a class A complex I ETC inhibitor, for example Mubritinib or a pharmaceutically acceptable salt thereof, for use in the treatment of a subject suffering from AML, wherein said AML exhibits low level of expression of one or more of the genes depicted in Table 2.

The present disclosure also provides a method for determining the likelihood that a subject suffering from AML responds to a treatment with a mitochondrial activity inhibitor, preferably a class A complex I ETC inhibitor, for example Mubritinib or a pharmaceutically acceptable salt thereof, the method comprising determining whether AML cells from said subject exhibit low level of expression of one or more of the genes depicted in Table 2, wherein low level of expression of the one or more genes in said AML cells is indicative that the subject has a high likelihood of responding to said treatment.

In an embodiment, at least one gene of Table 2 is weakly expressed (underexpressed) in the AML. In an embodiment, at least 2 genes of Table 2 are weakly expressed in the AML. In an embodiment, at least 3 genes of Table 2 are weakly expressed in the AML. In an embodiment, at least 4 genes of Table 2 are weakly expressed in the AML. In an embodiment, at least 5 genes of Table 2 are weakly expressed in the AML. In an embodiment, at least 10 genes of Table 2 are weakly expressed in the AML. In an embodiment, all the above-noted genes are upregulated in the AML. In an embodiment, at least 1, 2, 3, 4, 5, 10, or more of the genes listed in Table 2 are weakly expressed in the AML. In an embodiment, all the genes listed in Table 2 are weakly expressed in the AML.

In an embodiment, the above-mentioned method further comprises measuring the level of expression of one or more of the genes of Table 2 in a sample comprising leukemic cells from the subject, and comparing the level to a reference level or control to determine whether the one or more genes is/are weakly expressed or underexpressed in the leukemic cells, and wherein if the one or more genes is/are weakly expressed or underexpressed in the leukemic cells, selecting the subject for treatment with a mitochondrial activity inhibitor, preferably a class A complex I ETC inhibitor, for example Mubritinib or a pharmaceutically acceptable salt thereof.

The determination of the expression of the one or more genes or encoded gene products (e.g., mRNA, protein) disclosed herein (e.g., HOX-network genes, the genes in Tables 1 to 3) may be performed using any known methods to detect nucleic acids or proteins. In embodiments, the expression is compared to a control or reference level (e.g., the level obtained a sample from an AML subjects known to be resistant or sensitive to mitochondrial activity inhibitors (e.g., Mubritinib-resistant or Mubritinib-sensitive) to assess the subject's likelihood of responding to mitochondrial activity inhibitors (e.g., Mubritinib) and to determine whether the subject may be treated with a mitochondrial activity inhibitor, for example Mubritinib or a pharmaceutically acceptable salt thereof.

The levels of nucleic acids corresponding to the above-mentioned genes (e.g., transcripts) can then be evaluated according to commonly used methods such as those disclosed below, e.g., with or without the use of nucleic acid amplification methods. In some embodiments, nucleic acid amplification methods can be used to detect the level of expression of the one or more genes. For example, oligonucleotide primers and/or probes may be used in amplification and detection methods that use nucleic acid substrates isolated by any of a variety of well-known and established methodologies (e.g., Sambrook et al., supra; Lin et al., in Diagnostic Molecular Microbiology, Principles and Applications, pp. 605-16 (Persing et al., eds. (1993); Ausubel et al., Current Protocols in Molecular Biology (2001 and later updates thereto)). Methods for amplifying nucleic acids include, but are not limited to, for example the polymerase chain reaction (PCR) and reverse transcription PCR (RT-PCR) (see e.g., U.S. Pat. Nos. 4,683,195; 4,683,202; 4,800,159; 4,965,188), ligase chain reaction (LCR) (see, e.g., Weiss, Science 254: 1292-93 (1991)), strand displacement amplification (SDA) (see e.g., Walker et al., Proc. Natl. Acad. Sci. USA 89:392-396 (1992); U.S. Pat. Nos. 5,270,184 and 5,455,166), Thermophilic SDA (tSDA) (see e.g., European Pat. No. 0 684 315) and methods described in U.S. Pat. No. 5,130,238; Lizardi et al., BioTechnol. 6:1197-1202 (1988); Kwoh et al., Proc. Natl. Acad. Sci. USA 86:1173-77 (1989); Guatelli et al., Proc. Natl. Acad. Sci. USA 87:1874-78 (1990); U.S. Pat. Nos. 5,480,784; 5,399,491; U.S. Publication No. 2006/46265. The methods include the use of Transcription Mediated Amplification (TMA), which employs an RNA polymerase to produce multiple RNA transcripts of a target region (see, e.g., U.S. Pat. Nos. 5,480,784; 5,399,491 and U.S. Publication No. 2006/46265). The levels of nucleic acids may also be measured by “Next Generation Sequencing” (NGS) methods such as RNA sequencing (RNA-seq). In an embodiment, the method for measuring the level of expression of the one or more genes comprises a step of nucleic acid amplification.

The nucleic acid or amplification product may be detected or quantified by hybridizing a probe (e.g., a labeled probe) to a portion of the nucleic acid or amplified product. The primers and/or probes may be labelled with a detectable label that may be, for example, a fluorescent moiety, chemiluminescent moiety, radioisotope, biotin, avidin, enzyme, enzyme substrate, or other reactive group. As used herein, the term “detectable label” refers to a moiety emitting a signal (e.g., light) that may be detected using an appropriate detection system. Any suitable detectable label may be used in the method described herein. Detectable labels include, for example, enzyme or enzyme substrates, reactive groups, chromophores such as dyes or colored particles, luminescent moieties including bioluminescent, phosphorescent, or chemiluminescent moieties, and fluorescent moieties. In an embodiment, the detectable label is a fluorescent moiety. Fluorophores that are commonly used include, but are not limited to, fluorescein, 5-carboxyfluorescein (FAM), 2′7′-dimethoxy-4′5′-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), and 5-(2′-aminoethyl)aminonaphthalene-I-sulfonic acid (EDANS). The fluorophore may be any fluorophore known in the art, including, but not limited to: FAM, TET, HEX, Cy3, TMR, ROX, Texas Red®, LC red 640, Cy5, and LC red 705. Fluorophores for use in the methods and compositions provided herein may be obtained commercially, for example, from Biosearch Technologies (Novato, Calif.), Life Technologies (Carlsbad, Calif.), GE Healthcare (Piscataway N.J.), Integrated DNA Technologies (Coralville, Iowa) and Roche Applied Science (Indianapolis, Ind.). In some embodiments, the fluorophore is chosen to be usable with a specific detector, such as a specific spectrophotometric thermal cycler, depending on the light source of the instrument. In some embodiments, if the assay is designed for the detection of two or more target nucleic acids (multiplex assays), two or more different fluorophores may be chosen with absorption and emission wavelengths that are well separated from each other (i.e., have minimal spectral overlap). In some embodiments, the fluorophore is chosen to work well with one or more specific quenchers. A representative example of a suitable combination of fluorescent label and quenchers is FAM/ZEN/IBFQ, which comprises the fluorescent FAM (excitation max.=494 nm, emission max.=520 nm), the ZEN™ quencher (non-abbreviation; absorption max 532 nm), and the Iowa black fluorescein quencher (IBFQ, absorption max=531 nm) (Integrated DNA Technologies®). Covalent attachment of detectable label and/or quencher to primer and/or probe can be accomplished according to standard methodology well known in the art.

Other well-known detection techniques include, for example, gel filtration, gel electrophoresis and visualization of the amplicons, and High Performance Liquid Chromatography (HPLC). In certain embodiments, for example using real-time TMA or real-time PCR, the level of amplified product is detected as the product accumulates. In an embodiment, the method for measuring the level of expression of the one or more genes comprises a step of detection or quantification of a nucleic acid or amplification product with a probe.

In an embodiment, the above-mentioned method comprises a step of normalizing the gene expression levels, i.e. normalization of the measured levels of the above-noted genes against a stably expressed control gene (or housekeeping gene) to facilitate the comparison between different samples. “Normalizing” or “normalization” as used herein refers to the correction of raw gene expression values/data between different samples for sample to sample variations, to take into account differences in “extrinsic” parameters such as cellular input, nucleic acid (RNA) or protein quality, efficiency of reverse transcription (RT), amplification, labeling, purification, etc., i.e. differences not due to actual “intrinsic” variations in gene expression by the cells in the samples. Such normalization is performed by correcting the raw gene expression values/data for a test gene (or gene of interest) based on the gene expression values/data measured for one or more “housekeeping” or “control” genes, i.e. whose expressions are known to be constant (i.e. to show relatively low variability) between the cells of different tissues and under different experimental conditions. Thus, in an embodiment, the above-mentioned method further comprises measuring the level of expression of a housekeeping gene in the biological sample. Suitable housekeeping genes are known in the art and several examples are described in WO 2014/134728, including those depicted in Table 3 below.

TABLE 3
Examples of housekeeping genes

| Gene | RefSeq/GenBank Accession # |
|---|---|
| ABI1 | NM_001012750, NM_001012751, NM_001012752, |
| | NM_001178116, NM_001178119, NM_001178120, |
| | NM_001178121, NM_001178122, NM_001178123, |
| | NM_001178124, NM_001178125, NM_005470 |
| ACIN1 | NM_001164814, NM_001164815, NM_001164816, |
| | NM_001164817, NM_014977 |
| ACP1 | NM_001040649, NM_004300, NM_007099 |
| ADAR | NM_001025107, NM_001111, NM_001193495 |
| ADD1 | NM_001119, NM_014189, NM_014190, NM_176801 |
| ANAPC5 | NM_001137559, NM_016237 |
| ARF1 | NM_001024226, NM_001024227, NM_001024228, |
| | NM_001658 |
| ATP5B | NM_001686 |
| ATP6V1G1 | NM_004888 |
| ATXN2L | NM_007245, NM_017492, NM_145714, |
| | NM_148414, NM_148415, NM_148416 |
| AUP1 | NM_181575 |
| C1orf144 | NM_001114600, NM_015609 |
| C20orf43 | NM_016407 |
| C6orf62 | NM_030939 |
| CAPRIN1 | NM_005898, NM_203364 |
| CASC3 | NM_007359 |
| CCNI | NM_006835 |
| CDC37 | NM_007065 |
| CDV3 | NM_001134422, NM_001134423, NM_017548 |
| CMPK1 | NM_001136140, NM_016308 |
| CMTM3 | NM_144601, NM_181553 |
| COPB1 | NM_001144061, NM_001144062, NM_016451 |
| COPS5 | NM_006837 |
| CS | NM_004077 |
| CSDE1 | NM_001007553, NM_001130523, NM_001242891, |
| | NM_001242892, NM_001242893, NM_007158 |
| DAP3 | NM_001199849, NM_001199850, NM_001199851, |
| | NM_004632, NM_033657 |
| DCAF8 | NM_015726 |
| DDX5 | NM_004396 |
| DLST | NM_001933 |
| DNAJC7 | NM_001144766, NM_003315 |
| DOCK2 | NM_004946 |
| E2F4 | NM_001950 |
| EIF3I | NM_003757 |
| EIF4H | NM_022170, NM_031992 |
| EWSR1 | NM_001163285, NM_001163286, NM_001163287, |
| | NM_005243, NM_013986 |
| FAM32A | NM_014077 |
| GABARAPL2 | NM_007285 |
| GNB1 | NM_002074, NM_001282538, NM_001282539 |
| GORASP2 | NM_001201428, NM_015530 |
| GTF2F1 | NM_002096 |
| HDAC3 | NM_003883 |
| HNRNPA2B1 | NM_002137, NM_031243 |
| HNRNPC | NM_001077442, NM_001077443, NM_004500, |
| | NM_031314 |
| HNRNPD | NM_002138, NM_031369, NM_031370 |
| HNRNPH3 | NM_012207, NM_021644 |
| HNRNPK | NM_002140, NM_031262, NM_031263 |
| HNRNPL | NM_001005335, NM_001533 |
| HNRNPU | NM_004501, NM_031844 |
| HNRNPUL1 | NM_007040 |
| IDH3B | NM_001258384, NM_006899, NM_174855, |
| | NM_174856 |
| IK | NM_006083 |
| KARS | NM_001130089, NM_005548 |
| KHDRBS1 | NM_006559 |
| LSM14A | NM_001114093, NM_015578 |
| MAPRE1 | NM_012325 |
| MARS | NM_004990 |
| MLF2 | NM_005439 |
| MMADHC | NM_015702 |
| MORF4L1 | NM_001265605, NM_006791, NM_206839 |
| MRFAP1 | NM_033296 |
| MRPL9 | NM_031420 |
| MTA2 | NM_004739 |
| MYL12B | NM_001144944, NM_001144945, NM_033546 |
| NOL7 | NM_016167 |
| NRD1 | NM_001101662, NM_001242361, NM_002525 |
| OCIAD1 | NM_001079839, NM_001079840, NM_001079841, |
| | NM_001079842, NM_001168254, NM_017830 |
| PAPOLA | NM_001252006, NM_032632 |
| PCBP2 | NM_001098620, NM_001128911, NM_001128912, |
| | NM_001128913, NM_001128914, NM_005016, |
| | NM_031989 |
| POLR2C | NM_032940 |
| PSMA1 | NM_002786, NM_148976 |
| PSMB1 | NM_002793 |
| PSMD2 | NM_002808 |
| PSMD6 | NM_014814, NM_001271780, NM_001271779, |
| | NM_001271781 |
| PSMD7 | NM_002811 |
| PSME1 | NM_006263, NM_176783 |
| PSME3 | NM_001267045, NM_005789, NM_176863 |
| PSMF1 | NM_006814, NM_178578 |
| PTPRA | NM_002836, NM_080840, NM_080841 |
| RAB7A | NM_004637 |
| RBM22 | NM_018047 |
| RBM8A | NM_005105 |
| RHOA | NM_001664 |
| RNF114 | NM_018683 |
| RNF7 | NM_001201370, NM_014245, NM_183237 |
| SEC22B | NM_0048925 |
| SEC31A | NM_001077206, NM_001077207, NM_001077208, |
| | NM_001191049, NM_014933, NM_016211 |
| SERP1 | NM_014445 |
| SF3A1 | NM_001005409, NM_005877 |
| SF3B2 | NM_006842 |
| SLC25A3 | NM_002635, NM_005888, NM_213611 |
| SNW1 | NM_012245 |
| SON | NM_032195, NM_138927 |
| SRP14 | NM_003134 |
| SRPR | NM_001177842, NM_003139 |
| SRSF5 | NM_001039465, NM_006925 |
| SRSF9 | NM_003769 |
| SSR2 | NM_003145 |
| STX16 | NM_001001433, NM_001134772, NM_001134773, |
| | NM_001204868, NM_003763 |
| SUMO1 | NM_001005781, NM_001005782, NM_003352 |
| SUMO3 | NM_006936 |
| SUPT6H | NM_003170 |
| TCEB1 | NM_001204857, NM_001204858, NM_001204859, |
| | NM_001204860, NM_001204861, NM_001204862, |
| | NM_001204863, NM_001204864, NM_005648 |
| TH1L | NM_198976 |
| TMED2 | NM_006815 |
| TMEM50A | NM_014313 |
| TRIP12 | NM_004238 |
| U2AF1 | NM_001025203, NM_001025204, NM_006758 |
| UBE2D3 | NM_003340, NM_181886, NM_181887, |
| | NM_181888, NM_181889, NM_181890, |
| | NM_181891, NM_181892, NM_181893 |
| UBE2I | NM_003345, NM_194259, NM_194260, |
| | NM_194261 |
| UBE2Z | NM_023079 |
| UBQLN1 | NM_013438, NM_053067 |
| USP39 | NM_001256725, NM_001256726, NM_001256728, |
| | NM_006590 |
| USP4 | NM_001251877, NM_003363, NM_199443 |
| VCP | NM_007126 |
| VPS4A | NM_013245 |
| XRN2 | NM_012255 |
| YME1L1 | NM_014263, NM_139312 |
| ZC3H11A | NM_001271675, NM_014827 |
| ZNF207 | NM_001032293, NM_001098507, NM_003457 |

Other commonly used housekeeping genes include TBP, YWHAZ, PGK1, LDHA, ALDOA, HPRT1, SDHA, UBC, GAPDH, ACTB, G6PD, VIM, TUBA A, PFKP, B2M, GUSB, PGAM1 and HMBS.

In an embodiment, the method for measuring the level of expression of the one or more genes further comprises measuring the level of expression of one or more housekeeping genes in a biological sample from the subject.

In another embodiment, the expression of the one or more genes or encoded gene products is measured at the protein level. Methods to measure the amount/level of proteins are well known in the art. Protein levels may be detected directly using a ligand binding specifically to the protein, such as an antibody or a fragment thereof. In embodiments, such a binding molecule or reagent (e.g., antibody) is labeled/conjugated, e.g., radio-labeled, chromophore-labeled, fluorophore-labeled, or enzyme-labeled to facilitate detection and quantification of the complex (direct detection). Alternatively, protein levels may be detected indirectly, using a binding molecule or reagent, followed by the detection of the [protein/binding molecule or reagent] complex using a second ligand (or second binding molecule) specifically recognizing the binding molecule or reagent (indirect detection). Such a second ligand may be radio-labeled, chromophore-labeled, fluorophore-labeled, or enzyme-labeled to facilitate detection and quantification of the complex. Enzymes used for labeling antibodies for immunoassays are known in the art, and the most widely used are horseradish peroxidase (HRP) and alkaline phosphatase (AP). Examples of binding molecules or reagents include antibodies (monoclonal or polyclonal), natural or synthetic ligands, and the like.

Examples of methods to measure the amount/level of protein in a sample include, but are not limited to: Western blot, immunoblot, enzyme-linked immunosorbent assay (ELISA), “sandwich” immunoassays, radioimmunoassay (RIA), immunoprecipitation, surface plasmon resonance (SPR), chemiluminescence, fluorescent polarization, phosphorescence, immunohistochemical (IHC) analysis, matrix-assisted laser desorption/ionization time-of-flight (MALDI-TOF) mass spectrometry, microcytometry, microarray, antibody array, microscopy (e.g., electron microscopy), flow cytometry, proteomic-based assays, and assays based on a property or activity of the protein including but not limited to ligand binding or interaction with other protein partners, enzymatic activity, fluorescence. For example, if the protein of interest is a kinase known to phosphorylate of given target, the level or activity of the protein of interest may be determined by the measuring the level of phosphorylation of the target in the presence of the test compound. If the protein of interest is a transcription factor known to induce the expression of one or more given target gene(s), the level or activity of the protein of interest may be determined by the measuring the level of expression of the target gene(s).

“Control level” or “reference level” are used interchangeably herein and broadly refers to a separate baseline level measured in a comparable “control” sample, which is generally from a subject having a known level of expression of the gene of interest and/or whose responsiveness to a mitochondria activity inhibitor (e.g., Mubritinib) is known, for example an AML sample from a subject known to not respond to a mitochondria activity inhibitor (e.g., Mubritinib). The corresponding control level may be a level corresponding to an average or median level calculated based of the levels measured in several reference or control subjects (e.g., a pre-determined or established standard level). The control level may be a pre-determined “cut-off” value recognized in the art or established based on levels measured in samples from one or a group of control subjects (e.g., a group of subject known to not respond to a mitochondria activity inhibitor (e.g., Mubritinib)). For example, the “threshold reference level” may be the level corresponding the minimal level of expression (cut-off) of the gene(s) that permits to distinguish in a statistically significant manner AML subjects who are likely to respond to a mitochondria activity inhibitor (e.g., Mubritinib-sensitive) from those who are unlikely to respond to a mitochondria activity inhibitor (e.g., Mubritinib-resistant), which may be determined using samples from AML patients with different pharmalogical responses to a mitochondria activity inhibitor (e.g., Mubritinib), for example. Alternatively, the “threshold reference level” may be the level corresponding the level of gene expression (cut-off) that permits to best or optimally distinguish in a statistically significant manner AML subjects sensitive to a mitochondria activity inhibitor (e.g., Mubritinib) from AML subjects resistant a mitochondria activity inhibitor (e.g., Mubritinib). The corresponding reference/control level may be adjusted or normalized for age, gender, race, or other parameters. The “control level” can thus be a single number/value, equally applicable to every subject individually, or the control level can vary, according to specific subpopulations of patients. Thus, for example, older men might have a different control level than younger men, and women might have a different control level than men. The predetermined standard level can be arranged, for example, where a tested population is divided equally (or unequally) into groups, such as a low-likelihood group, a medium-likelihood group and/or a high-likelihood group or into quadrants or quintiles. It will also be understood that the control levels according to the invention may be, in addition to predetermined levels or standards, levels measured in other samples (e.g. from a subject having a known level of expression of the gene of interest and/or whose responsiveness to Mubritinib is known) tested in parallel with the experimental sample. The reference or control levels may correspond to normalized levels, i.e. reference or control values subjected to normalization based on the expression of a housekeeping gene.

“Higher expression” or “higher level of expression” (overexpression) as used herein refers to (i) higher expression of the one or more of the above-mentioned genes (protein and/or mRNA) in one or more given cells present in the sample (relative to the control) and/or (ii) higher amount of cells expressing the one or more genes in the sample (relative to the control). “Low/weak expression” or “lower/weaker level of expression” (underexpression) as used herein refers to (i) lower expression of the one or more genes (protein and/or mRNA) in one or more given cells present in the sample (relative to the control) and/or (ii) lower amount of cells expressing the one or more genes in the sample (relative to the control). In an embodiment, higher or lower refers to a level of expression that is above or below the control level (e.g., the predetermined cut-off value). In another embodiment, higher or lower refers to a level of expression that is at least one standard deviation above or below the control level (e.g., the predetermined cut-off value) (e.g. that is statistically significant as determined using a suitable statistical analysis), and a “similar expression” or “similar level of expression” refers to a level of expression that is less than one standard deviation above or below the control level (e.g., the predetermined cut-off value) (e.g. that is not statistically significant as determined using a suitable statistical analysis, such as False Discovery Rate (FDR)/q-values, Student t-test/p values, Mann-Whitney test/p values). In embodiments, higher or lower refers to a level of expression that is at least 1.5, 2, 2.5, 3, 4 or 5 standard deviations above or below the control level (e.g., the predetermined cut-off value. In another embodiment, “higher expression” refers to an expression that is at least 10, 20, 30, 40, 45 or 50% higher in the test sample relative to the control level. In an embodiment, “lower expression” refers to an expression that is at least 10, 20, 25, 30, 35, 40, 45, or 50% lower in the test sample relative to the control level. In another embodiment, higher or lower refers to a level of expression that is at least 1.5, 2-, 5-, 10-, 25-, or 50-fold higher or lower in the test sample relative to the control sample.

In another aspect, the present disclosure provides a method for treating AML, said method comprising administering to a subject in need thereof an effective amount of a mitochondria activity inhibitor (e.g., Mubritinib or a pharmaceutically acceptable salt thereof), wherein said AML exhibits one or more of the following cytogenetic or molecular risk factor: Normal Karyotype (NK), mutated NPM1, mutated CEBPA (e.g., mono- or bi-allelic), mutated FLT3, mutated DNMT3A, mutated TET2, mutated IDH1, mutated IDH2, mutated RUNX1, mutated WT1, mutated SRSF2, intermediate cytogenetic risk, intermediate cytogenetic risk with abnormal karyotype (intern(abnK)), trisomy 8 (+8) and abnormal chr(5/7). In another aspect, the present disclosure provides a use of a mitochondria activity inhibitor, preferably a class A complex I ETC inhibitor (e.g., Mubritinib or a pharmaceutically acceptable salt thereof) for treating a subject suffering from AML, wherein said AML exhibits one or more of the above-noted cytogenetic or molecular risk factors. In another aspect, the present disclosure provides a use of a mitochondria activity inhibitor, preferably a class A complex I ETC inhibitor (e.g., Mubritinib or a pharmaceutically acceptable salt thereof) for the manufacture of a medicament for treating a subject suffering from AML, wherein said AML exhibits one or more of the above-noted cytogenetic or molecular risk factors. In another aspect, the present disclosure provides a mitochondria activity inhibitor, preferably a class A complex I ETC inhibitor (e.g., Mubritinib or a pharmaceutically acceptable salt thereof) for use in the treatment of AML, wherein said AML exhibits one or more of the above-noted cytogenetic or molecular risk factors.

In an embodiment, the subject suffers from AML characterized by intermediate cytogenetic risk, Normal Karyotype (NK), and/or high HOX expression. In an embodiment, the subject suffers from AML with intermediate cytogenetic risk. In an embodiment, the subject suffers from AML with Normal Karyotype (NK). In an embodiment, the subject suffers from AML with high HOX expression.

The present disclosure also provides a method for determining the likelihood that a subject suffering from AML responds to a treatment with a mitochondria activity inhibitor, preferably a class A complex I ETC inhibitor (e.g., Mubritinib or a pharmaceutically acceptable salt thereof), the method comprising determining whether AML cells from said subject exhibit one or more of the following cytogenetic or molecular risk factor: Normal Karyotype (NK), mutated NPM1, mutated CEBPA, mutated FLT3, mutated DNMT3A, mutated TET2, mutated IDH1, mutated IDH2, mutated RUNX1, mutated WT1, mutated SRSF2, intermediate cytogenetic risk, intermediate cytogenetic risk with abnormal karyotype (intern(abnK)), trisomy 8 (+8) and abnormal chr(5/7), wherein the presence of said one or more of cytogenetic or molecular risk factor in said AML cells is indicative that the subject has a high likelihood of responding to said treatment. In an embodiment, the mutation in the above-noted mutated genes is at one or more of the positions depicted in Table 5B. In a further embodiment, the mutation in the above-noted mutated genes is one or more of the mutations depicted in Table 5B.

In an embodiment, the method/use is for treating NK-AML. In an embodiment, the method/use is for treating AML with mutated FLT3 (e.g., FLT3 with internal tandem duplications, FLT3-ITD). In an embodiment, the method/use is for treating AML with mutated CEBPA. In an embodiment, the method/use is for treating AML with mutated DNMT3A. In an embodiment, the method/use is for treating AML with mutated TET2. In an embodiment, the method/use is for treating AML with mutated IDH1. In an embodiment, the method/use is for treating AML with mutated IDH2. In an embodiment, the method/use is for treating AML with mutated RUNX1. In an embodiment, the method/use is for treating AML with mutated WT1. In an embodiment, the method/use is for treating AML with mutated SRSF2. In an embodiment, the method/use is for treating AML with intermediate cytogenetic risk with abnormal karyotype (intern(abnK)). In an embodiment, the method/use is for treating AML with trisomy 8 (+8). In an embodiment, the method/use is for treating AML with abnormal chromosome (5/7).

In an embodiment, the method is for treatment AML with any combination of 2, 3, 4, 5 or more of the above-mentioned cytogenetic or molecular risk factors.

In an embodiment, the AML is not AML with MLL translocations. In an embodiment, the AML is not EVI1-rearranged AML. In an embodiment, the AML is not Core Binding Factor (CBF) AML, for example AML with t(8:21) or inv(16) chromosomal rearrangements. In an embodiment, the AML is not AML with mutated NRAS, mutated c-KIT and/or mutated TP53.

In an embodiment, the AML is NK-AML with mutated NPM1. In an embodiment, the AML cells comprise a mutated NPM1, a mutated FLT3 (e.g., FLT3 with internal tandem duplications, FLT3-ITD) and a mutated DNA methylation gene such as DNMT3A. In a further embodiment, the AML cells comprise a mutated NPM1, a mutated FLT3 (e.g., FLT3 with internal tandem duplications, FLT3-ITD), a mutated DNA methylation gene such as DNMT3A, and do not comprise a mutated NRAS.

The cytogenetic and molecular risk factors defined herein are based on the 2008 World Health Organization (WHO) classification (Vardiman et al., Blood 2009 114:937-951), and recent advances in genomic classification (Papaemmanuil, E. et al. N Engl J Med 374, 2209-2221, 2016).

In another embodiment, the above-noted method/use further comprises determining the presence (or absence) of one or more of the cytogenetic and molecular risk factors defined herein in a sample comprising leukemic cells from the subject, wherein if one or more of the cytogenetic and molecular risk factors are present, selecting the subject for treatment with a mitochondrial activity inhibitor, preferably a class A complex I ETC inhibitor, e.g., Mubritinib or a pharmaceutically acceptable salt thereof.

Methods and kits to identify cytogenetic or molecular risk factors (mutation(s), translocations, fusions, chromosomal abnormalities, etc.) are well known in the art, and include, for example, karyotype, fluorescence in situ hybridization (FISH), reverse transcription polymerase chain reaction (RT-PCR), DNA sequencing, and microarray technology (see, e.g., Gulley et al., J Mol Diagn. 2010 January; 12(1): 3-16). The determination of the presence of the mutation(s), translocations, fusions, etc. in the sample may be performed using any suitable methods (see, e.g., Syvanen, Nat Rev Genet. 2001 December; 2(12):930-42). For example, the presence of the mutation(s) may be detected at the genomic DNA, transcript (RNA or cDNA) or protein level. Examples of suitable methods for determining sequences and polymorphisms at the nucleic acid level include sequencing of the nucleic acid sequence encompassing the mutation(s), e.g., in the genomic DNA or transcript (cDNA), for example by “Next Generation Sequencing” methods (e.g., genome sequencing, RNA sequencing (RNA-seq)) or other sequencing methods; hybridization of a nucleic acid probe capable of specifically hybridizing to a nucleic acid sequence comprising the mutation(s) and not to (or to a lesser extent to) a corresponding nucleic acid sequence that does not comprises the mutation(s) (under comparable hybridization conditions, such as stringent hybridization conditions) (e.g., molecular beacons); restriction fragment length polymorphism analysis (RFLP); Amplified fragment length polymorphism PCR (AFLP-PCR); amplification of a nucleic acid fragment comprising the mutation(s) using a primer specifically hybridizing to a nucleic acid sequence comprising the mutation(s), wherein the primer produces an amplified product if the mutation(s) is/are present and does not produce the same amplified product when a nucleic acid sequence not comprising the mutation(s) is used as a template for amplification, nucleic acid sequence based amplification (Nasba), primer extension assay, FLAP endonuclease assay (Invader assay, Olivier M. (2005). Mutat Res. 573(1-2):103-10), 5′-nuclease assay (McGuigan F. E. and Ralston S. H. (2002) Psychiatr Genet. 12(3):133-6), oligonucleotide ligase assay. Other methods include in situ hybridization analyses and single-stranded conformational polymorphism analyses. Several SNP genotyping platforms are commercially available. Additional methods will be apparent to one of skill in the art.

The determination of the presence of the mutation(s) and/or fusion(s) may also be achieved at the polypeptide/protein level. Examples of suitable methods for detecting alterations at the polypeptide level include sequencing of the encoded polypeptide; digestion of the encoded polypeptide followed by mass spectrometry or HPLC analysis of the peptide fragments, wherein the mutated polypeptide results in an altered mass spectrometry or HPLC spectrum as compared to the unmutated polypeptide; and immunodetection using an immunological reagent (e.g., an antibody, a ligand) which exhibits altered immunoreactivity with a mutated polypeptide relative to a corresponding unmutated polypeptide. Immunodetection can measure the amount of binding between a polypeptide molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots, and other techniques known to those of ordinary skill in the art (see Harlow and Lane, Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). Methods to generate antibodies exhibiting altered immunoreactivity with a mutated polypeptide relative to a corresponding unmutated polypeptide are described in more detail below.

All these detection techniques may also be employed in the format of microarrays, e.g., SNP microarrays, DNA microarrays, protein-arrays, antibody microarrays, tissue microarrays, electronic biochip or protein-chip based technologies (see Schena M., Microarray Biochip Technology, Eaton Publishing, Natick, Mass., 2000).

In another embodiment, the methods described herein to identify the presence of one or more features in the AML cells further comprise obtaining or collecting a biological sample from a subject. In various embodiments, the sample can be from any source that contains biological material suitable for the detection of the mutation(s), such as genomic DNA, RNA (cDNA), and/or proteins, for example a tissue or cell sample from the subject (blood cells, immune cells (e.g., lymphocytes), etc. that comprises leukemic cells (AML cells). The sample may be subjected to cell purification/enrichment techniques to obtain a cell population enriched in a specific cell subpopulation or cell type(s). The sample may be subjected to commonly used isolation and/or purification techniques for enrichment in nucleic acids (genomic DNA, cDNA, mRNA) and/or proteins. Accordingly, in an embodiment, the method may be performed on an isolated nucleic acid and/or protein sample, such as isolated genomic DNA. The biological sample may be collected using any methods for collection of biological fluid, tissue or cell sample, such as venous puncture for collection of blood cell samples.

The present disclosure also provides a method for treating AML, said method comprising administering to a subject in need thereof an effective amount of a mitochondrial activity inhibitor, preferably a class A complex I ETC inhibitor, e.g., Mubritinib or a pharmaceutically acceptable salt thereof, wherein said AML exhibits a leukemic stem cell (LSC) frequency of about 1 LSC per 1×10⁶ total cells, or more.

The present disclosure also provides a method for determining the likelihood that a subject suffering from AML responds to a treatment with a mitochondrial activity inhibitor, preferably a class A complex I ETC inhibitor, e.g., Mubritinib or a pharmaceutically acceptable salt thereof, the method comprising determining LSC frequency in the AML cells from said subject, wherein an LSC frequency of about 1 LSC per 1×10⁶ total cells, or more, is indicative that the subject has a high likelihood of responding to said treatment.

In an embodiment, the AML exhibits an LSC frequency of about 1 LSC per 9×10⁵ total cells, or more. In an embodiment, the AML exhibits an LSC frequency of about 1 LSC per 8×10⁵ total cells, or more. In an embodiment, the AML exhibits an LSC frequency of about 1 LSC per 7×10⁵ total cells, or more. In an embodiment, the AML exhibits an LSC frequency of about 1 LSC per 6×10⁵ total cells, or more. In an embodiment, the AML exhibits an LSC frequency of about 1 LSC per 5×10⁵ total cells, or more. In an embodiment, the AML exhibits an LSC frequency of about 1 LSC per 4×10⁵ total cells, or more. In an embodiment, the AML exhibits an LSC frequency of about 1 LSC per 3×10⁵ total cells, or more. In an embodiment, the AML exhibits an LSC frequency of about 1 LSC per 2×10⁵ total cells, or more. In an embodiment, the AML exhibits an LSC frequency of about 1 LSC per 1×10⁵ total cells, or more.

LSC frequency in an AML cell sample may be measured using methods known in the art, for example using a flow cytometric-based assay using LSC-associated markers (CD34+CD38⁻) and light scatter aberrancies (Terwijn et al., PLoS One. 2014 Sep. 22; 9(9):e107587) or using a limiting dilution assay (LDA) in a model of xenotransplantation based on NOD/SCID/IL2Ryc-deficient (NSG) mice (Sarry et al., J Clin Invest. 2011; 121(1):384-395; Pabst, C. et al. Blood 127, 2018-2027 and US Patent Publication No. 2014/0343051 A1)), as used in the studies described below. In an embodiment, the LSC frequency is as measured using the limiting dilution assay in NSG mice described in the Examples below.

In an embodiment, the above-mentioned method further comprises measuring the LSC frequency in a sample comprising leukemic cells from the subject, and wherein if the LSC frequency is about 1 LSC per 1×10⁶ total cells, or more, selecting the subject for treatment with Mubritinib or a pharmaceutically acceptable salt thereof.

In another embodiment, the mitochondrial activity inhibitor, preferably a class A complex I ETC inhibitor, e.g., Mubritinib or pharmaceutically acceptable salt thereof, is administered/used as a prodrug, for example as a pharmaceutically acceptable ester. The term “prodrug” refers to analogs of an active agent (mitochondrial activity inhibitor such as Mubritinib or pharmaceutically acceptable salt thereof) that are pharmacologically acceptable and substantially non-toxic to the subject to which they are administered. More specifically, the prodrug retains the biological effectiveness and properties of the active agent (e.g., Mubritinib or pharmaceutically acceptable salt thereof) and when absorbed into the bloodstream of a warm-blooded animal, is cleaved or metabolized in such a manner as to produce the parent active agent. Methods to produce prodrugs of compounds are known in the art.

In the methods of the present disclosure, the mitochondrial activity inhibitor, preferably a class A complex I ETC inhibitor, e.g., Mubritinib or pharmaceutically acceptable salt thereof, may be administered using any conventional route, for example orally, intravenously, parenterally, subcutaneously, intramuscularly, intraperitoneally, intranasally or pulmonary (e.g., aerosol).

In an embodiment, the mitochondrial activity inhibitor, preferably a class A complex I ETC inhibitor, e.g., Mubritinib or pharmaceutically acceptable salt thereof, is present in a pharmaceutical composition. Thus, in an another aspect, the present invention provides a composition for use in the treatment of AML in a subject, the composition comprising a mitochondrial activity inhibitor, preferably a class A complex I ETC inhibitor such as Mubritinib or pharmaceutically acceptable salt thereof.

In an embodiment, the composition comprises one or more pharmaceutically acceptable carriers or excipients. Supplementary active compounds can also be incorporated into the compositions. The carrier/excipient can be suitable, for example, for intravenous, parenteral, subcutaneous, intramuscular, intraperitoneal, intranasal or pulmonary (e.g., aerosol) administration (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). Pharmaceutical compositions may be prepared using standard methods known in the art by mixing the Mubritinib or pharmaceutically acceptable salt thereof having the desired degree of purity with one or more optional pharmaceutically acceptable carriers and/or excipients.

The term “pharmaceutically acceptable carrier or excipient” as used herein has its normal meaning in the art and refers any ingredient that is not an active ingredient (mitochondrial activity inhibitor such as Mubritinib or pharmaceutically acceptable salt thereof) itself that does not interfere with effectiveness of the biological activity of the active ingredient and that is not toxic to the subject, i.e., is a type of carrier or excipient and/or is for use in an amount which is not toxic to the subject. Excipients/carriers include for example binders, lubricants, diluents, fillers, thickening agents, disintegrants/dissolution promoting agents, plasticizers, coatings, barrier layer formulations, lubricants, surfactants, stabilizing agent, release-delaying agents, permeation enhancers, glidants, anti-caking agents, anti-tacking agents, stabilizing agents, anti-static agents, swelling agents and other components. As those of skill would recognize, a single excipient can fulfill more than two functions at once, e.g., can act as both a binding agent and a thickening agent. As those of skill will also recognize, these terms are not necessarily mutually exclusive.

Useful diluents, e.g., fillers, include, for example and without limitation, dicalcium phosphate, calcium diphosphate, calcium carbonate, calcium sulfate, lactose, cellulose, kaolin, sodium chloride, starches, powdered sugar, colloidal silicon dioxide, titanium oxide, alumina, talc, colloidal silica, microcrystalline cellulose, silicified micro crystalline cellulose and combinations thereof. Fillers that can add bulk to tablets with minimal drug dosage to produce tablets of adequate size and weight include croscarmellose sodium NF/EP (e.g., Ac-Di-Sol™); anhydrous lactose NF/EP (e.g., Pharmatose™ DCL 21); and/or povidone USP/EP.

Binder materials include, for example and without limitation, starches (including corn starch and pregelatinized starch), gelatin, sugars (including sucrose, glucose, dextrose and lactose), polyethylene glycol, povidone, waxes, and natural and synthetic gums, e.g., acacia sodium alginate, polyvinylpyrrolidone, cellulosic polymers (e.g., hydroxypropyl cellulose, hydroxypropyl methylcellulose, methyl cellulose, hydroxyethyl cellulose, carboxymethylcellulose, colloidal silicon dioxide NF/EP (e.g., Cab-O-Sil™ M5P), Silicified Microcrystalline Cellulose (SMCC), e.g., Silicified microcrystalline cellulose NF/EP (e.g., Prosolv™ SMCC 90), and silicon dioxide, mixtures thereof, and the like), veegum, and combinations thereof.

Useful lubricants include, for example, canola oil, glyceryl palmitostearate, hydrogenated vegetable oil (type I), magnesium oxide, magnesium stearate, mineral oil, poloxamer, polyethylene glycol, sodium lauryl sulfate, sodium stearate fumarate, stearic acid, talc and, zinc stearate, glyceryl behapate, magnesium lauryl sulfate, boric acid, sodium benzoate, sodium acetate, sodium benzoate/sodium acetate (in combination), DL-leucine, calcium stearate, sodium stearyl fumarate, mixtures thereof, and the like.

Bulking agents include, for example: microcrystalline cellulose, for example, AVICEL® (FMC Corp.) or EMCOCEL® (Mendell Inc.), which also has binder properties; dicalcium phosphate, for example, EMCOMPRESS® (Mendell Inc.); calcium sulfate, for example, COMPACTROL® (Mendell Inc.); and starches, for example, Starch 1500; and polyethylene glycols (CARBOWAX®).

Disintegrating or dissolution promoting agents include: starches, clays, celluloses, alginates, gums, crosslinked polymers, colloidal silicon dioxide, osmogens, mixtures thereof, and the like, such as crosslinked sodium carboxymethyl cellulose (AC-DI-SOL), sodium croscarmelose, sodium starch glycolate (EXPLOTAB®, PRIMO JEL) crosslinked polyvinylpolypyrrolidone (PLASONE-XL), sodium chloride, sucrose, lactose and mannitol.

Antiadherents and glidants employable in the core and/or a coating of the solid oral dosage form may include talc, starches (e.g., cornstarch), celluloses, silicon dioxide, sodium lauryl sulfate, colloidal silica dioxide, and metallic stearates, among others.

Examples of silica flow conditioners include colloidal silicon dioxide, magnesium aluminum silicate and guar gum.

Suitable surfactants include pharmaceutically acceptable non-ionic, ionic and anionic surfactants. An example of a surfactant is sodium lauryl sulfate. If desired, the pharmaceutical composition to be administered may also contain minor amounts of nontoxic auxiliary substances such as wetting or emulsifying agents, pH-buffering agents and the like, for example, sodium acetate, sorbitan monolaurate, triethanolamine sodium acetate, triethanolamine oleate, etc. If desired, flavoring, coloring and/or sweetening agents may be added as well.

Examples of stabilizing agents include acacia, albumin, polyvinyl alcohol, alginic acid, bentonite, dicalcium phosphate, carboxymethylcellulose, hydroxypropylcellulose, colloidal silicon dioxide, cyclodextrins, glyceryl monostearate, hydroxypropyl methylcellulose, magnesium trisilicate, magnesium aluminum silicate, propylene glycol, propylene glycol alginate, sodium alginate, carnauba wax, xanthan gum, starch, stearate(s), stearic acid, stearic monoglyceride and stearyl alcohol.

Examples of thickening agent can be for example talc USP/EP, a natural gum, such as guar gum or gum arabic, or a cellulose derivative such as microcrystalline cellulose NF/EP (e.g., Avicel™ PH 102), methylcellulose, ethylcellulose or hydroxyethylcellulose. A useful thickening agent is hydroxypropyl methylcellulose, an adjuvant which is available in various viscosity grades.

Examples of plasticizers include: acetylated monoglycerides; these can be used as food additives; Alkyl citrates, used in food packagings, medical products, cosmetics and children toys; Triethyl citrate (TEC); Acetyl triethyl citrate (ATEC), higher boiling point and lower volatility than TEC; Tributyl citrate (TBC); Acetyl tributyl citrate (ATBC), compatible with PVC and vinyl chloride copolymers; Trioctyl citrate (TOC), also used for gums and controlled release medicines; Acetyl trioctyl citrate (ATOC); Trihexyl citrate (THC), compatible with PVC, also used for controlled release medicines; Acetyl trihexyl citrate (ATHC), compatible with PVC; Butyryl trihexyl citrate (BTHC, trihexyl o-butyryl citrate), compatible with PVC; Trimethyl citrate (TMC), compatible with PVC; alkyl sulphonic acid phenyl ester, polyethylene glycol (PEG) or any combination thereof. Optionally, the plasticizer can comprise triethyl citrate NF/EP.

Examples of permeation enhancers include: sulphoxides (such as dimethylsulphoxide, DMSO), azones (e.g. laurocapram), pyrrolidones (for example 2-pyrrolidone, 2P), alcohols and alkanols (ethanol, or decanol), glycols (for example propylene glycol and polyethylene glycol), surfactants and terpenes.

In an embodiment, the pharmaceutical composition is an oral formulation, is for oral administration. In an embodiment, the pharmaceutical composition is in the form of a tablet or pill.

Formulations suitable for oral administration may include (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Any suitable amount of the mitochondrial activity inhibitor, preferably a class A complex I ETC inhibitor, e.g., Mubritinib or pharmaceutically acceptable salt thereof, or pharmaceutical composition comprising same, may be administered to a subject. The dosages will depend on many factors including the mode of administration. Typically, the amount of mitochondrial activity inhibitor, e.g., Mubritinib or pharmaceutically acceptable salt thereof, contained within a single dose will be an amount that effectively prevent, delay or treat AML without inducing significant toxicity.

For the prevention, treatment or reduction in the severity of AML, the appropriate dosage of the mitochondrial activity inhibitor, preferably a class A complex I ETC inhibitor, e.g., Mubritinib or pharmaceutically acceptable salt thereof, or composition will depend, for example, on the type of AML to be treated, the severity and course of the AML, whether the mitochondrial activity inhibitor or composition is administered for preventive or therapeutic purposes, previous therapy, the patient's clinical history and response to the mitochondrial activity inhibitor or composition, and the discretion of the attending physician. The mitochondrial activity inhibitor, e.g., Mubritinib or pharmaceutically acceptable salt thereof, or composition may be suitably administered to the patient at one time or over a series of treatments. Preferably, it is desirable to determine the dose-response curve in vitro, and then in useful animal models, prior to testing in humans. The present disclosure provides dosages for the mitochondrial activity inhibitor, e.g., Mubritinib or pharmaceutically acceptable salt thereof, and compositions comprising same. For example, depending on the type and severity of the AML, about 1 μg/kg to about 1000 mg per kg (mg/kg) of body weight of the mitochondrial activity inhibitor may be administered per day. In embodiments, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, and may increase by 25 mg/kg increments up to 1000 mg/kg, or may range between any two of the foregoing values. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays. The mitochondrial activity inhibitor, e.g., Mubritinib or pharmaceutically acceptable salt thereof, may be administered according to any suitable dosing schedule/regimen, e.g., twice-a-day, once-a-day, every 2 days, twice-a-week, weekly, etc. In an embodiment, the administration is once-a-day.

In an embodiment, the pharmaceutical composition comprises from about 0.1 mg to about 100 mg of the mitochondrial activity inhibitor, e.g., Mubritinib or pharmaceutically acceptable salt thereof. In further embodiments, the pharmaceutical composition comprises from about 1 mg to about 50 mg, from about 2 mg to about 30 mg, from about 5 mg to about 25 or 20 mg, or about 10 mg, of the mitochondrial activity inhibitor, preferably a class A complex I ETC inhibitor, e.g., Mubritinib or pharmaceutically acceptable salt thereof.

In an embodiment, the above-mentioned treatment comprises the use/administration of more than one (i.e. a combination of) active/therapeutic agent or therapy, one of which being with a mitochondrial activity inhibitor, e.g., Mubritinib or pharmaceutically acceptable salt thereof. The combination of prophylactic/therapeutic agents and/or compositions of the present disclosure may be administered or co-administered (e.g., consecutively, simultaneously, at different times) in any conventional dosage form. Co-administration in the context of the present disclosure refers to the administration of more than one therapeutic in the course of a coordinated treatment to achieve an improved clinical outcome. Such co-administration may also be coextensive, that is, occurring during overlapping periods of time. For example, a first agent (e.g., a mitochondrial activity inhibitor such as Mubritinib or pharmaceutically acceptable salt thereof) may be administered to a patient before, concomitantly, before and after, or after a second active agent or therapy is administered. The agents may in an embodiment be combined/formulated in a single composition and thus administered at the same time. In an embodiment, the one or more active agent(s) is used/administered in combination with one or more agent(s) currently used to prevent or treat the disorder in question, for example chemotherapeutic drugs used for the treatment of AML. Mubritinib may also be used in combination with other AML therapy, for example stem cell/bone marrow transplantation.

In another aspect, the present disclosure provides a method for determining whether a subject suffering from AML is likely to respond to a treatment with a mitochondrial activity inhibitor, for example Mubritinib or a pharmaceutically acceptable salt thereof, said method comprising determining whether the AML has at least one of the following features: (a) high level of expression of one or more homeobox (HOX)-network genes; (b) high level of expression of one or more of the genes depicted in Table 1; (c) low level of expression of one or more of the genes depicted in Table 2; (d) one or more of the following cytogenetic or molecular risk factor: intermediate cytogenetic risk, Normal Karyotype (NK), mutated NPM1, mutated CEBPA, mutated FLT3, mutated DNA methylation genes, mutated RUNX1, mutated WT1, mutated SRSF2, intermediate cytogenetic risk with abnormal karyotype (intern(abnK)), trisomy 8 (+8) and abnormal chr(5/7); and (e) a leukemic stem cell (LSC) frequency of about 1 LSC per 1×10⁶ total cells, or more; wherein the presence of at least one of these features in said AML is indicative that the patient is likely to respond to a treatment with a mitochondrial activity inhibitor.

Methods to measure the above-noted features are well known in the art, and representative methods are described above.

### MODE(S) FOR CARRYING OUT THE INVENTION

The present invention is illustrated in further details by the following non-limiting examples.

### Example 1: Materials and Methods

Human Leukemia Samples

This study was approved by the Research Ethics Boards (REB) of Universite de Montreal, Maisonneuve-Rosemont Hospital and Charles Lemoyne Hospital (Longueuil, QC, Canada). All AML samples were collected with an informed consent between 2001 and 2017 according to the procedures of the Banque de Cellules Leucemiques du Québec (BCLQ). Umbilical cord blood (CB) units were collected from consenting mothers and human CD34⁺ CB cells were isolated with the EasySep® positive selection kit (StemCell Technologies, Vancouver, Canada, catalog number 18056) (Fares et al., Science. 2014 Sep. 19; 345(6203):1509-12).

Chemical Screen

Primary Cells:

frozen AML mono-nucleated cells were thawed at 37° C. in Iscove's modified Dulbecco's medium (IMDM) containing 20% FBS and DNase 1 (100 μg/mL). Cells were then cultured in optimized AML growth medium as previously reported (Pabst, C. et al. Nature methods 11, 436-442, 2014): IMDM, 15% BIT (bovine serum albumin, insulin, transferrin; Stem Cell Technologies®), 100 ng/mL SCF, 50 ng/mL FLT3-L, 20 ng/mL IL-3, 20 ng/mL G-CSF (Shenandoah®), 10⁻⁴ M β-mercaptoethanol, 500 nM SR1 (Alichem®), 500 nM UM729 (synthesized at the Medicinal Chemistry Core Facility at the Institute for Research in Immunology and Cancer (IRIC)), gentamicin (50 μg/mL) and ciprofloxacin (10 μg/mL).

Expanded CB cells are cells that resulted from 6 days of UM171-supplemented culture of fresh CB cells, as described in Fares et al., supra. Fresh CB cells are cells directly collected from umbilical cord blood specimens, following a positive CD34 selection (EasySep® kit, StemCell Technologies, Vancouver, Canada), as described in Fares et al., Science. 2014 Sep. 19; 345(6203):1509-12. CB cells (fresh and expanded) were cultivated in StemSpan®-ACF (Stemcell Technologies 09855) containing SCF 100 ng/mL, TPO 100 ng/mL, FLT3-L 50 ng/mL (Shenandoah®), Glutamax® 1×, LDL 10 μg/mL, ciprofloxacin 10 μg/mL, 500 nM SR1 (Alichem®) and 35 nM UM171 (synthesized at the Medicinal Chemistry Core Facility at the Institute for Research in Immunology and Cancer (IRIC)).

Cell Lines.

OCI-AML3 cells were maintained in alpha-MEM, 20% FBS whereas breat tumor BT474 cells were maintained in DMEM 10% FBS. BT474 (ATCC® HTB-20™) was a kind gift from the laboratory of Sylvie Mader, whereas OCI-AML3 and OCI-AML5 cells were purchased from the German cell bank (DSMZ, accession Nos. ACC 582 and ACC 247, respectively).

Compounds.

All powders were dissolved in DMSO and diluted in culture medium immediately before use. Final DMSO concentration in all conditions was 0.1%. The 60 compounds were purchased from various suppliers including Santa Cruz, Selleckchem, Calbiochem, AdooQ Bioscience, Cayman chemical and Synkinase.

Cell Dose-Response Viability Assays.

Patient cells were seeded in 384-well plates at a density of 5,000 cells in 50 μL per well. In the initial screen, compounds were added to seeded cells in serial dilutions (10 dilutions, 1:3, starting from 10 μM or 20 μM), in duplicates. Cells treated with 0.1% DMSO without additional compound were used as negative controls. Viable cell counts per well were evaluated after 6 days of culture using the CellTiterGlo® assay (Promega®) according to the manufacturer's instruction. The percent of inhibition was calculated as follows: 100-(100×(mean luminescence(compound)/mean luminescence(DMSO)); where mean-luminescence(compound) corresponds to the average of luminescent signals obtained for the compound-treated cells, and mean-luminescence(DMSO) corresponds to the average of luminescent signals obtained for the control DMSO-treated cells.

EC₅₀ values (corresponding to the concentration of compound required to reach 50% of inhibition) were calculated using ActivityBase® SARview Suite (IDBS, London, UK) and GraphPad® Prism 4.03 (La Jolla, Calif., USA) by four-parameter-non-linear curve fitting methods.

Leukemic Stem Cell (LSC) Frequency Assessment.

LSC frequencies were assessed in immunocompromised NSG mice using limiting dilution assays, as detailed previously (Pabst, C. et al. Blood 127, 2018-2027, 2016). NOD.Cg-Prkdc^{scid} II2rg^{tm1Wjl}/SzJ (NSG) mice were purchased from Jackson Laboratory® (Bar Harbor, Me.) and bred in a pathogen-free animal facility. All AML samples were transplanted via the tail vein into 8 to 12-week old sublethally irradiated (250 cGy, ¹³⁷Cs-gamma source) NSG mice. AML cells were transplanted at four different cell doses in groups of four recipient mice directly after thawing. Human leukemic engraftment in mouse bone marrow was determined by flow cytometry between 10 and 16 weeks post-transplant. On average 150,000 gated events were acquired. Mice were considered positive if human cells represented >1% of the bone marrow cell population. Mice were excluded only in case of obvious non-leukemia related death (e.g., first two weeks after irradiation). To discriminate between engraftment of leukemic and normal cells present in unsorted patient samples only recipients with proportions of CD45⁺CD33⁺ or CD45⁺CD34⁺ cells higher than proportions of CD19⁺CD33⁻ or CD3⁺ were considered to harbor cells of leukemic origin.

Flow Cytometry Staining

The following FACS antibodies were used: anti-human CD45 Pacific Blue (BioLegend 304029), CD45 fluorescein isothiocyanate (FITC; BioLegend 304006), CD33 phycoerythrin (PE; BD Bioscience 555450), CD33 BV421 (BD 562854), CD34 antigen-presenting cell (APC; BD Bioscience 555824), CD34 APC (Stem Cell Technologies 10613), CD3 FITC (BD Bioscience 555332), CD4 APC-Cy7 (BD 560158), CD8 APC (BD 555369), CD3 PE-Cy5 (BD 555334), CD19 PE-Cy7 (BD Bioscience 557835), anti-mouse CD45.1 APC-efluor730 (eBioscience 47-0453-82), ERBB2-PE (Biolegend®, 324406), Annexin V FITC (BD Bioscience®, 556419). Dead cells were stained using Propidium iodide at a final concentration of lpg/mL. For ROS quantification cells were stained with 1 μM H2DCFDA (Thermo Fisher, D399) under normal growth conditions. Cells were analyzed on LSRII® flow cytometer (BD Bioscience®), BD Canto® II cytometer (BD Bioscience) or on an IQue Screener (Intellicyt®) and results were analyzed with BD fluorescence-activated cell sorter (FACS) Diva® 4.1 and FlowJo® X software.

Next-Generation Sequencing and Mutation Quantification

Workflow for sequencing, mutation analysis and transcripts quantification have been described previously (Lavallee, V.-P. et al. Blood 125, 140-143, 2014; Lavallee, V.-P. et al. Nat. Genet. 47, 1030-1037, 2015). Briefly, libraries were constructed with TruSeq® RNA/TruSeq® DNA Sample Preparation Kits (Illumina®). Sequencing was performed using an Illumina® HiSeq 2000 with 200 cycles paired end runs. Sequence data were mapped to the reference genome hg19 using the Illumina® Casava 1.8.2 package and Elandv2 mapping software according to RefSeq annotations (UCSC, Apr. 16, 2014). Variants were identified using Casava 1.8.2 and fusions or larger mutations such as partial tandem duplications with Tophat 2.0.7 and Cufflinks 2.1.1.

Transcript levels are given as Reads Per Kilobase per Million mapped reads (RPKM) and genes are annotated according to RefSeq annotations (UCSC, Apr. 16, 2014).

LC/MS Metabolite Measurements (Citric Acid Cycle Intermediates and Glutathione)

LC/MS metabolite measurements were carried out at the McGill Metabolomics platform. Authentic metabolite standards were purchased from Sigma-Aldrich Co., while the following LC/MS grade solvents and additives were purchased from Fisher: ammonium acetate, formic acid, water, methanol, and acetonitrile. OCI-AML3 cells (5 million cells, quadruplicates, treated with either DMSO or Mubritinib 500 nM for 20 h) were washed twice with ice-cold 150 mM ammonium formate pH 7.2. Metabolites were then extracted using 380 μl of LC/MS grade 50% methanol/50% water mixture and 220 μl of cold acetonitrile. Samples were then homogenized by 1.4 mm ceramic bead beating 2 min at 30 Hz (TissueLyser, Qiagen). A volume of 300 μl of ice-cold water and 600 μl of ice-cold methylene chloride were added to the lysates. Samples were vortexed and allowed to rest on ice for 10 min for phase separation followed by centrifugation at 4,000 rpm for 5 min. The upper aqueous layer was transferred to a fresh pre-chilled tube. Samples were eventually dried by vacuum centrifugation operating at −4° C. (Labconco) and stored at −80° C. until ready for LC-MS/MS data collection. For LC-MS/MS analysis, specimens were first re-suspended in 50 μl of water and clarified by centrifugation for 15 min at 15,000 rpm at 1° C. Samples were maintained at 4° C. for the duration of the LC-MS/MS analysis in the autosampler. Specimens were separated by U-HPLC (Ultra-High Performance Liquid Chromatography) (1290 Infinity, Agilent Technologies) using a Scherzo SM-C18 (3 mm×150 mm) 3 μm column and guard column (Imtakt USA) operating at 10° C.

Seahorse Metabolic Flux Experiments

Oxygen consumption rates and extracellular acidification rates were measured using a 96-well Seahorse Bioanalyzer XFe96® or XFe24® according to the manufacturer's instructions (Agilent Technologies). Seahorse XF Base medium was supplemented with 1 mM pyruvate, 2 mM glutamine and 10 mM glucose in the case of Mitochondrial Stress Test and with 1 mM pyruvate, 2 mM glutamine and no glucose in the case of Glycolytic Stress Test. The pH of the Seahorse media was then adjusted at 7.4 prior to assay. In brief, leukemic cells were seeded into Seahorse 96-well (or 24-well) plates pre-coated for 3 h with poly-lysine (Sigma-Aldrich, P4707) at a density of 75,000 cells/well in 100 μL (or 150,000 cells/well, in 150 μL) of temperature/CO₂ pre-adjusted Seahorse media per well. The Seahorse plates were then centrifuged at 1400 rpm for 5 min. An additional 75 μL (or 375 μL) of Seahorse media was then added and cells were eventually analyzed following the manufacturer's instructions by adding compounds in a constant volume of 25 μL (or 75 μL). Compounds were acutely injected in cells at a final concentration of 1 μM for Mubritinib, 1 μM for Oligomycin, 0.5 μM for FCCP, 0.5 μM for Rotenone/Antimycin A, Glucose 10 mM and 2-Deoxy-Glucose 50 mM.

Cell-Free Assay for ETC Complex I Activity

The cell-free kit assay for ETC complex I activity was purchased from MitoSciences (Abcam, Cambridge, UK), and used in accordance with the manufacturer's protocol. IC₅₀ values were calculated using GraphPad® Prism 4.03 (La Jolla, Calif., USA) by four-parameter-non-linear curve fitting methods.

Statistics

Analysis of differential gene expression was performed using the Wilcoxon rank-sum test and the false discovery rate (FDR) method was applied for global gene analysis as previously described (Lavallee, V.-P. et al. Blood 125, 140-143, 2014). Differential overall survival p-values were calculated by log-rank test on patients belonging to the prognostic Leucegene cohort.

### Example 2: HOX-High AML Patients Generally have a Poor Disease Prognosis

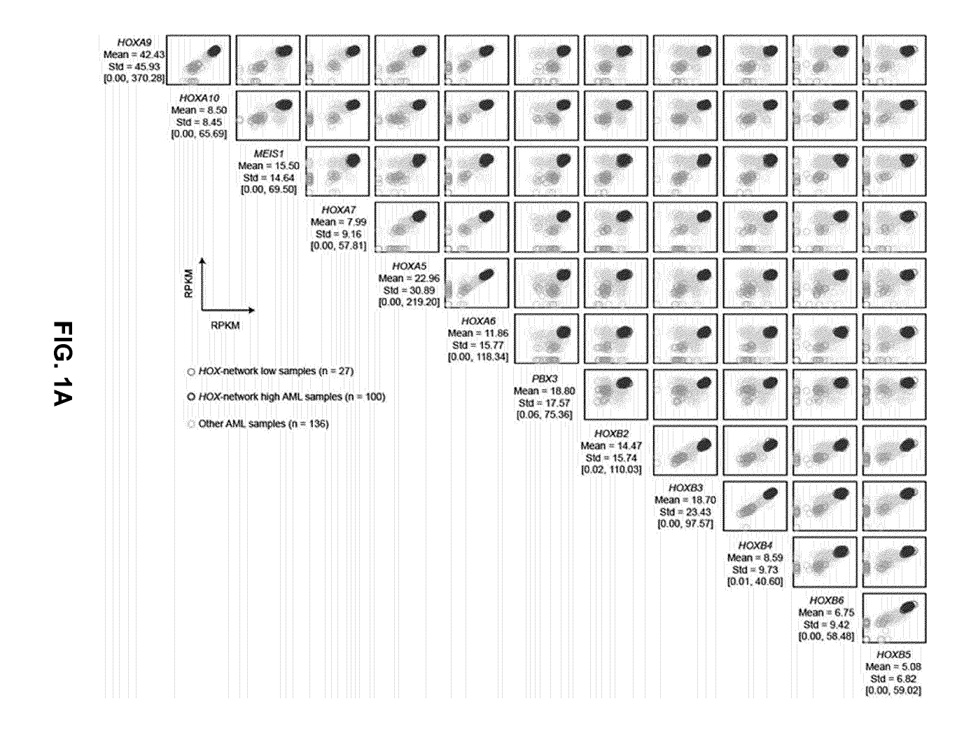

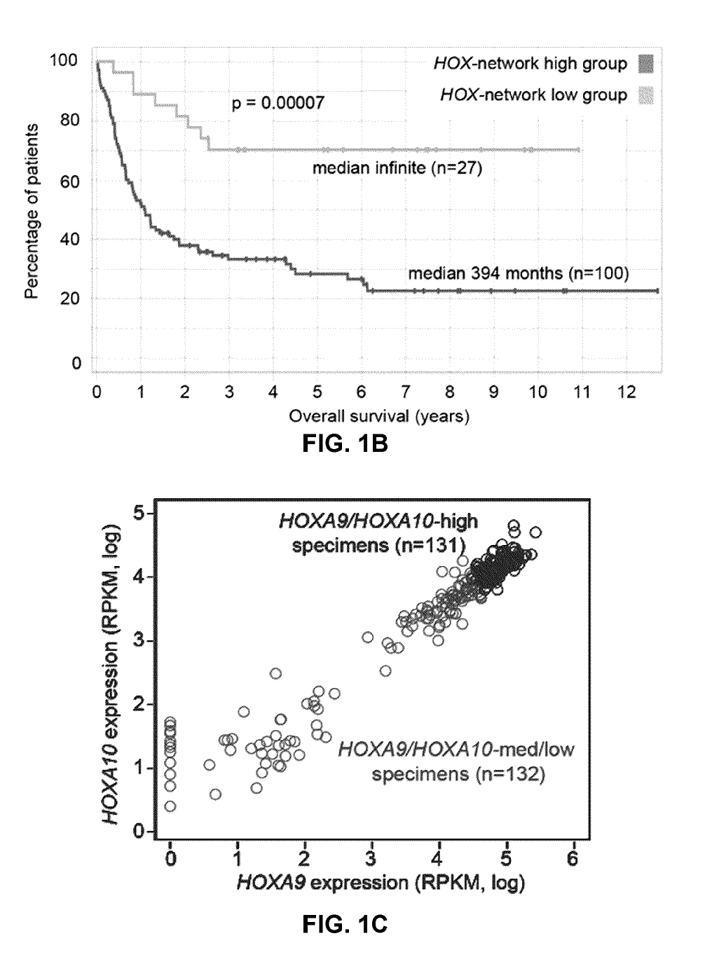

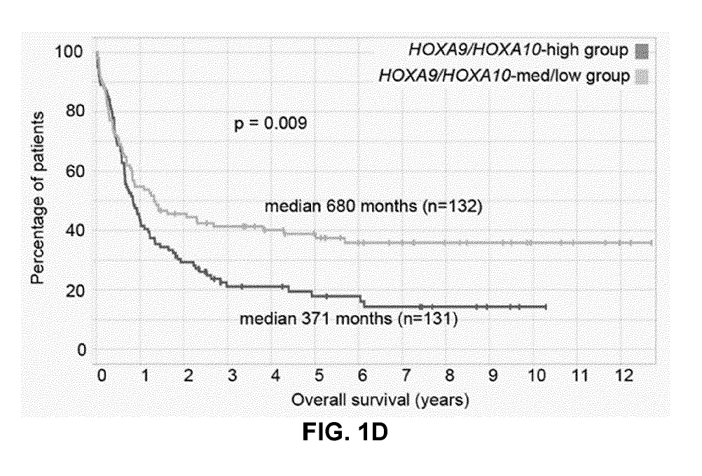

Consistent high expression of genes belonging to the HOX network (FIG. 1A) in AML cell samples was shown to be associated with a significantly decreased patient overall survival (FIG. 1B). FIG. 1C shows the correlation between HOXA9 and HOXA10 expression in leukemic cells, and FIG. 1D show that survival of AML patients belonging to the HOXA9/HOXA10 high group is similar to that of patients belonging to HOX-network high subgroup shown in FIG. 1B, indicating that high expression of HOXA9 and HOXA10 may be used as a surrogate for the detection of HOX network-high patients. Overall, these data show that patients having AML cells exhibiting high expression of HOX-network genes have a poor prognosis and would benefit from suitable AML treatments.

### Example 3: Mubritinib Efficiently Inhibits HOX-High AML Cells

Using high expression of HOXA9 and HOXA10 as a surrogate for the detection of HOX-network-high patient samples, the survival of HOX-high versus HOX-med/low specimens contacted with 60 inhibitors targeting receptor tyrosine kinases (RTK), members of the RAS and PI3K pathways, was assessed (FIG. 2A, Table 4).

TABLE 4
Results of the chemical screen on HOX-high versus HOX-med/low specimens

| | | | | MEAN | FOLD |
|---|---|---|---|---|---|
| | | | MEAN | EC50 | CHANGE |
| | DRUG MAIN | MEDIAN | EC50 | HOX- | High/Med- |
| DRUG NAME | TARGET | P-VALUE | HOX-high | med/low | low |
| AZD8055 | mTOR | 0.000457283 | 323.6 | 69.2 | 4.7 |
| AZD2014 | mTOR | 0.000136596 | 782.5 | 217.1 | 3.6 |
| OSI027 | mTOR | 2.64323E−05 | 2031.5 | 665.8 | 3.1 |
| Dasatinib | Bcr-ABL | 0.10101613 | 773.9 | 321.9 | 2.4 |
| MK2206 | AKT | 0.021880547 | 2279.4 | 1035.1 | 2.2 |
| E7080 Lenvatinib. | VEGFR2/3 | 0.038208282 | 7288.0 | 3341.1 | 2.2 |
| PDGFR Tyrosine | PDGFR and | 0.005614497 | 5995.5 | 2836.5 | 2.1 |
| Kinase Inhibitor VII | | | | | |
| Rapamycin | mTOR | 0.368763009 | 1888.4 | 951.3 | 2.0 |
| Triciribine AKT | PKB/AKT | 0.095563375 | 396.5 | 213.0 | 1.9 |
| inhibitor V | | | | | |
| GDC0941 | Pi3Kα/δ | 0.04358574 | 1107.5 | 602.6 | 1.8 |
| BYL719 | Pi3Kα | 0.01888386 | 7517.7 | 4749.7 | 1.6 |
| Masitinib | c-kit and | 0.012879581 | 7008.9 | 4565.0 | 1.5 |
| | PDGFR | | | | |
| SB590885 | B-RAF | 0.04957192 | 5882.5 | 3875.5 | 1.5 |
| MK2a Inhibitor | MK2a | 0.186736825 | 4942.1 | 3294.4 | 1.5 |
| Trametinib | MEK | 0.368763009 | 25.3 | 18.0 | 1.4 |
| GSK1120212 | | | | | |
| GS1101 idelalisib | Pi3Kδ | 0.04358574 | 7472.7 | 5666.7 | 1.3 |
| CAL101 | | | | | |
| Tanespimycin | HSP90 | 0.186736825 | 313.7 | 248.9 | 1.3 |
| 17AAG | | | | | |
| Sorafenib Tosylate | RAF-1 | 0.630575319 | 1097.6 | 935.6 | 1.2 |
| Vemurafenib | B-RAFV600E | 0.125276966 | 9704.1 | 8435.5 | 1.2 |
| ZM336372 | c-raf | 0.474218852 | 10000.0 | 8878.4 | 1.1 |
| cFMS Receptor | cFMS | 0.315495673 | 8859.1 | 7973.8 | 1.1 |
| Inhibitor III | | | | | |
| Ki8751 | VEGFR2 | 1 | 843.5 | 768.3 | 1.1 |
| U0126 | MEK | 0.706315606 | 4475.8 | 4110.4 | 1.1 |
| Imatinib Mesylate | Bcr-ABL and | 0.427163867 | 7927.4 | 7281.5 | 1.1 |
| OSI906 Linsitinib. | IGF1R and | 0.785012686 | 4253.0 | 3935.5 | 1.1 |
| | InsR | | | | |
| cFMS Receptor | cFMS | 0.412091118 | 9990.0 | 9308.7 | 1.1 |
| Inhibitor II | | | | | |
| AGL2043 | PDGFR | 0.315495673 | 8876.9 | 8381.7 | 1.1 |
| cFMS Receptor | cFMS | 0.825239723 | 10000.0 | 9574.4 | 1.0 |
| Inhibitor IV | | | | | |
| Nilotinib | Bcr-ABL | 0.968922491 | 3917.8 | 3811.6 | 1.0 |
| cMet Kinase | c-MET | 0.490500213 | 9980.0 | 9784.9 | 1.0 |
| Inhibitor III | | | | | |
| SU1498 | VEGFR | 0.224565264 | 9860.8 | 9672.9 | 1.0 |
| ERK Inhibitor II | ERK1 and 2 | 0.278965719 | 10000.0 | 10000.0 | 1.0 |
| FR180204 | | | | | |
| SB.203580 | p38 MAPK | 0.290816169 | 10000.0 | 10000.0 | 1.0 |
| Tie2 Kinase | Tie2 | 0.341480003 | 10000.0 | 10000.0 | 1.0 |
| Inhibitor | | | | | |
| LY303511 | neg control | 0.541053049 | 10000.0 | 10000.0 | 1.0 |
| SAR245408 | Pi3Kα/δ/γ | 0.649195464 | 10000.0 | 10000.0 | 1.0 |
| XL147 | | | | | |
| PI3K Inhibitor II | PI 3-Kγ | 0.765100706 | 10000.0 | 10000.0 | 1.0 |
| PD98059 | MEK | 0.412091118 | 9990.0 | 10000.0 | 1.0 |
| Enzastaurin | AKT | 0.490500213 | 9841.1 | 10000.0 | 1.0 |
| NVPAEW541 | IGFR | 0.989638561 | 1734.3 | 1766.9 | 1.0 |
| PD158780 | EGFR | 0.668032401 | 7465.2 | 7713.0 | 1.0 |
| AMD3100 | CXCR4 | 0.558451337 | 9588.2 | 10000.0 | 1.0 |
| VX702 | p38MAPK | 0.594023931 | 9549.9 | 10000.0 | 1.0 |
| PD173074 | FGFR and | 0.668032401 | 4947.1 | 5201.3 | 1.0 |
| | VEGFR | | | | |
| Erlotinib | EGFR | 0.490500213 | 8657.5 | 9143.5 | 0.9 |
| Selumetinib | MEK | 0.412091118 | 1240.2 | 1383.9 | 0.9 |
| AZD6244. | | | | | |
| Lapatinib | ErbB2 and | 0.278965719 | 8868.0 | 9923.5 | 0.9 |
| ditosylate. | | | | | |
| Gefitinib | EGFR | 0.033393019 | 8545.5 | 9697.7 | 0.9 |
| CI1040 | MEK | 0.745339369 | 2435.1 | 2771.9 | 0.9 |
| PD.184352. | | | | | |
| NVPBHG712 | EPHb4 and | 0.490500213 | 4440.1 | 5207.9 | 0.9 |
| p38 MAP Kinase | p38 MAPK | 0.063563244 | 7309.9 | 8788.0 | 0.8 |
| Inhibitor V | | | | | |
| R428 | AXL | 0.382887601 | 487.3 | 591.9 | 0.8 |
| Lenalidomide | TNFa and | 0.412091118 | 7309.9 | 9910.9 | 0.7 |
| | IKZF1 and 2 | | | | |
| Tipifarnib | RAS (H, N) | 0.125276966 | 109.2 | 157.5 | 0.7 |
| cMet RON Dual | c-MET and | 0.003925742 | 6740.6 | 9784.9 | 0.7 |
| Kinase Inhibitor | | | | | |
| Lonafarnib | RAS (H, K, N) | 0.118832336 | 2494.3 | 3668.1 | 0.7 |
| Flt3 Inhibitor IV | Flt3 | 0.169649847 | 3121.4 | 5031.1 | 0.6 |
| EGFR Inhibitor | EGFR | 0.169649847 | 185.8 | 300.5 | 0.6 |
| Quizartinib | flt3 | 0.095563375 | 229.5 | 520.1 | 0.4 |
| Mubritinib | ErbB2 | 0.010986372 | 315.0 | 1375.1 | 0.2 |

When compared to HOX-medllow samples, HOX-high patient cells were significantly more sensitive to the RTK ERBB2 inhibitor Mubritinib (FIG. 2B). No statistically significant difference in sensitivity was observed for the other compounds tested.

Dose response validation screening in a large cohort of AML samples confirmed that HOX-high patient cells are significantly more sensitive to Mubritinib than HOX-med/low AML cells (FIGS. 2C-D). Median Mubritinib EC₅₀ in the AML population tested was approximately 375 nM (FIG. 2E). Patients belonging to the Mubritinib-sensitive group (EC₅₀<375 nM) have a significantly decreased overall survival relative to patients of the Mubritinib-resistant group (EC₅₀≥375 nM, FIG. 2F). Specimens belonging to the Mubritinib-sensitive group overexpress genes of the HOX network (FIG. 2G), consistent with the results presented in Example 2. The most differentially expressed genes, (6-fold difference in RPKM values, RPKM>0.1) are shown in FIG. 2H and the complete Mubritinib sensitivity transcriptomic signature in AML is displayed in FIG. 2I, Table 1 (genes overexpressed more than 5-fold in RPKM values in Mubritinib-sensitive cells, RPKM>0.1) and Table 2 (genes underexpressed more than 5-fold in RPKM values in Mubritinib-sensitive cells, RPKM>0.1). Taken together, these data demonstrate that Mubritinib is a good candidate drug for the treatment of AML patients with high expression of HOX-network genes.

### Example 4: Mubritinib Target Population According to Classical Genetic/Cytogenetic Classifications

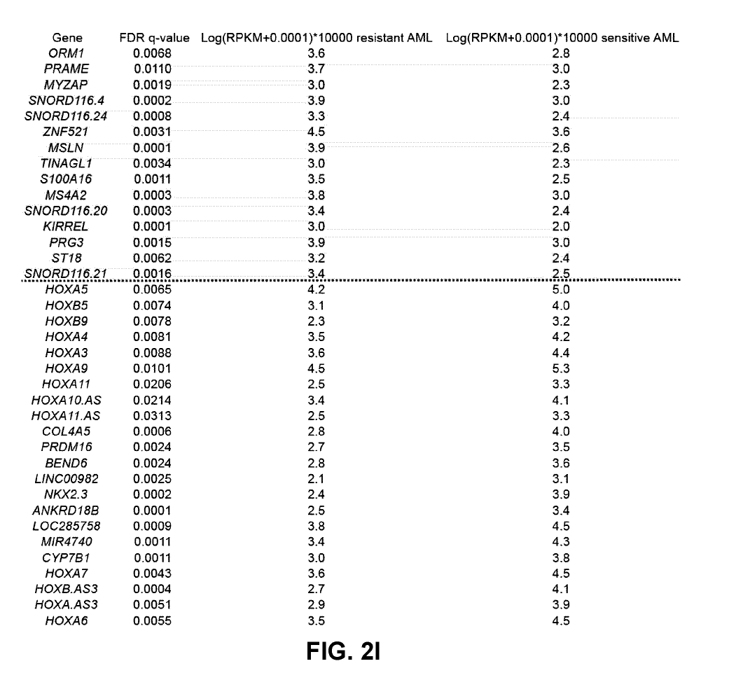

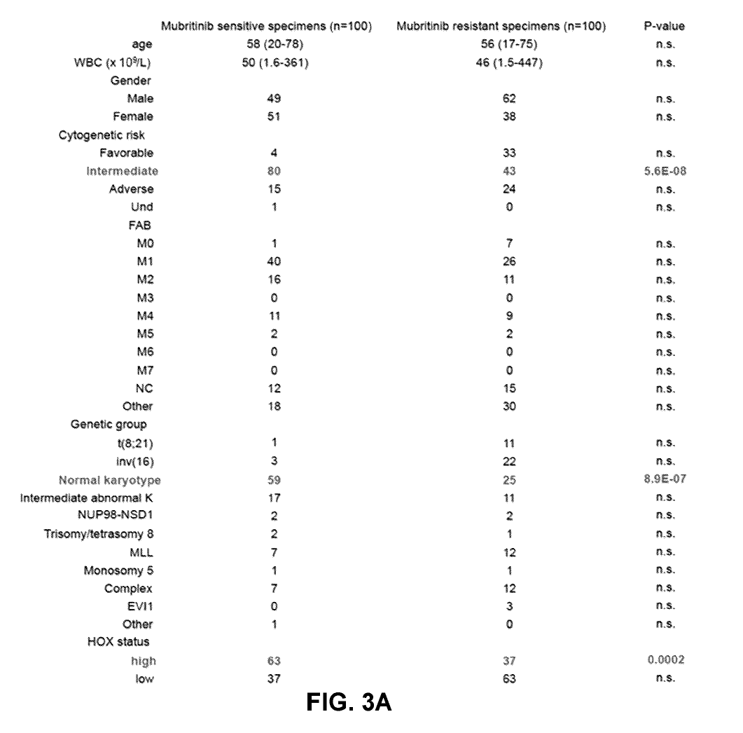

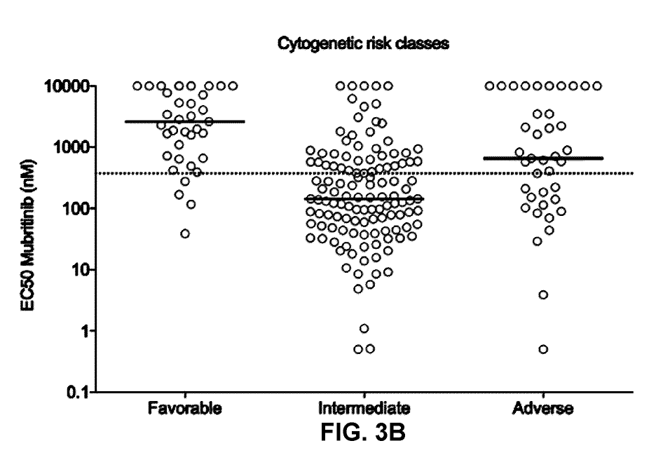

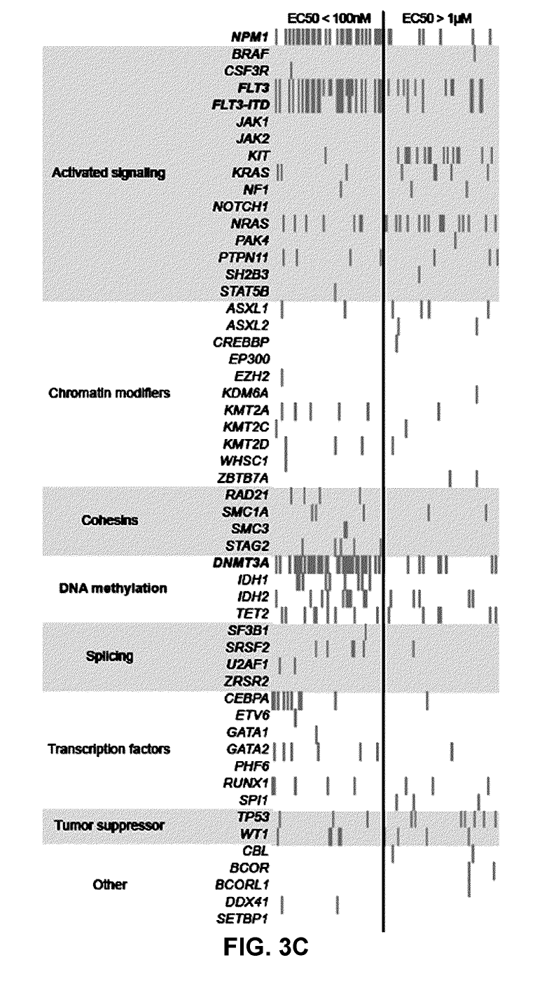

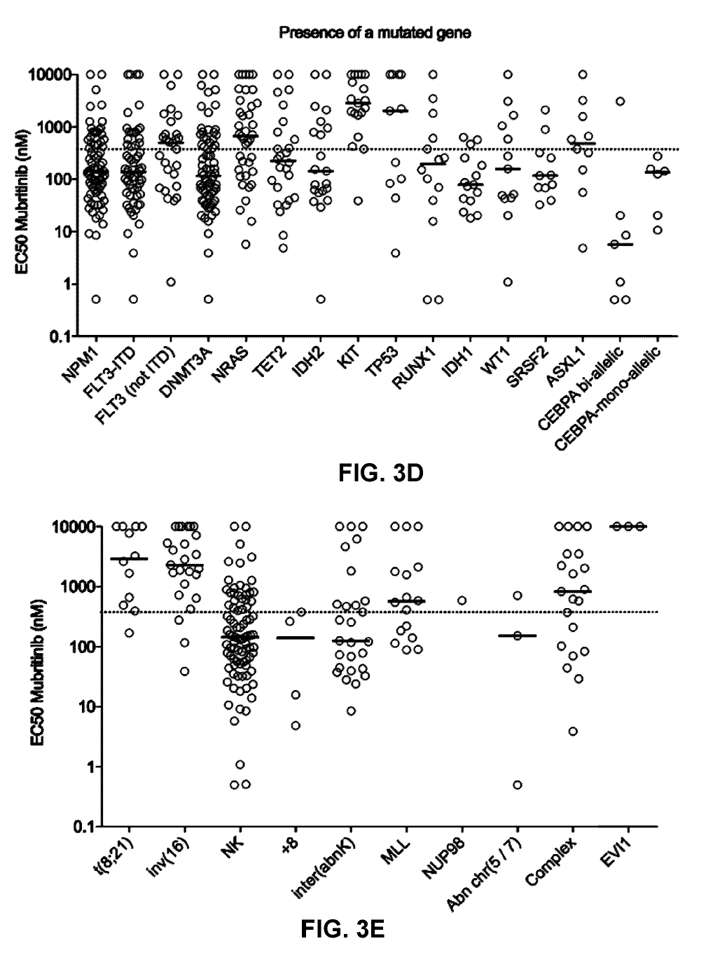

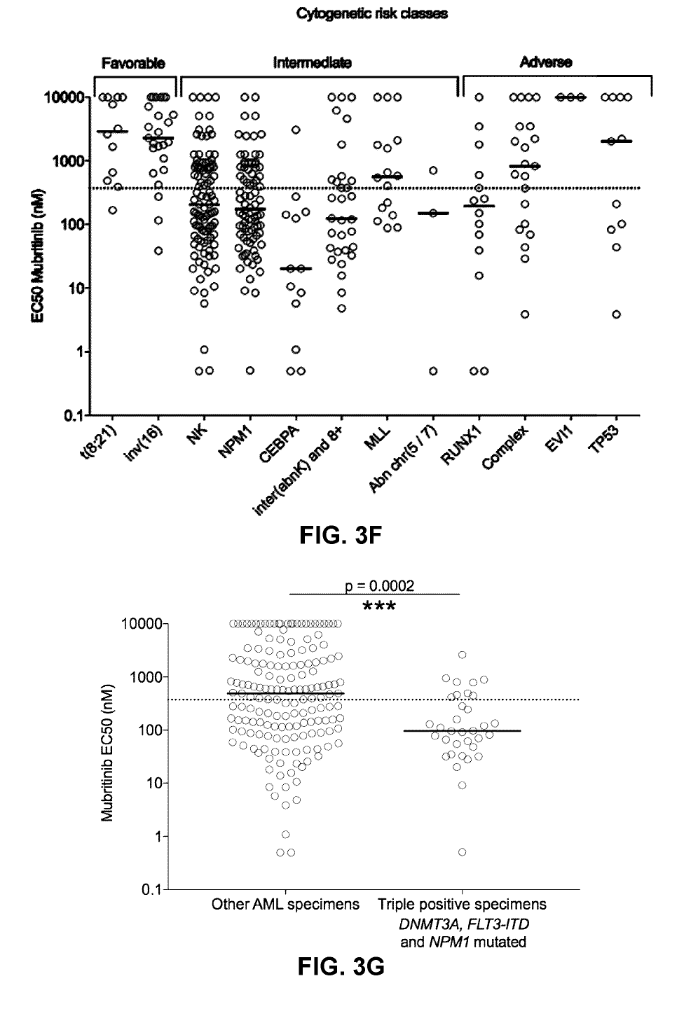

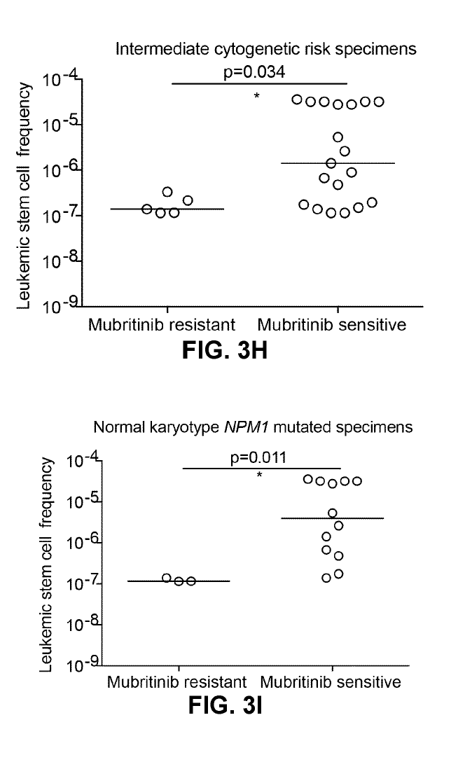

The results of a set of experiments aimed at identifying AML subtypes more sensitive to Mubritinib are depicted in FIGS. 3A-G and Tables 5A-5B. FIGS. 3A, 3B and 3F show that Mubritinib-sensitive AML cells most frequently belong to the intermediate cytogenetic risk class, often carry mutations in either NPM1, IDH1, SRSF2, CEBPA, DNMT3A or FLT3-ITD (FIGS. 3C, 3D and 3G), and generally have a normal karyotype (NK, FIGS. 3A, 3E and 3F). The most significantly enriched mutated in Mubritinib-sensitive versus resistant specimens were IDH1, SRSF2 and CEBPA, whereas AML samples mutations in NRAS, KIT, FLT3 not ITD and TP53 were underrepresented in the Mubritinib-sensitive group relative to the Mubritinib-resistant group (Table 5A). The detail of the mutations is depicted in Table 5B.

TABLE 5A
List of patient sample fold-enrichment according to the presence of a mutated
gene in Mubritinib-sensitive versus Mubritinib-resistant AML samples.

| | Genes mutated | | | | | | |
|---|---|---|---|---|---|---|---|
| | | FLT3- | FLT3 (not | | | | |
| | NPM1 | ITD | ITD) | DNMT3A | NRAS | TET2 | IDH2 |
| Mubritinib sensitive | 36.6 | 30.3 | 8.5 | 31.0 | 10.6 | 11.3 | 9.2 |
| (%) | | | | | | | |
| Mubritinib resistant | 22.4 | 17.8 | 15.0 | 24.3 | 22.4 | 9.3 | 7.5 |
| (%) | | | | | | | |
| Fold enrichment | 1.6 | 1.7 | 0.6 | 1.3 | 0.5 | 1.2 | 1.2 |
| sensitive/resistant | | | | | | | |

| | Genes mutated | | | | | | | |
|---|---|---|---|---|---|---|---|---|
| | KIT | TP53 | RUNX1 | IDH1 | WT1 | SRSF2 | ASXL1 | CEBPA |
| Mubritinib sensitive | 0.7 | 3.5 | 6.3 | 8.5 | 5.6 | 6.3 | 2.8 | 8.5 |
| (%) | | | | | | | | |
| Mubritinib resistant | 16.8 | 5.6 | 4.7 | 2.8 | 4.7 | 1.9 | 5.6 | 0.9 |
| (%) | | | | | | | | |
| Fold enrichment | 0.0 | 0.6 | 1.4 | 3.0 | 1.2 | 3.4 | 0.5 | 9.0 |
| sensitive/resistant | | | | | | | | |

TABLE 5B
Detail of the mutations present in the AML samples.

| ASXL1 | CEBPA | DNMT3A | FLT3 | IDH1 | IDH2 | KIT |
|---|---|---|---|---|---|---|
| R417* | L219P | K906Q | 575- | R132C | R172K | D419- |
| A472- | E215* | G890S | A680V | R132G | R140Q | D812V |
| G643- | R204- | L888M | D586- | R132H | R140L | D812Y |
| G645- | L196- | R882H | D593- | R132S | | L416- |
| L696- | V195- | R882P | D600- | | | N818K |
| Q708* | K194- | R882C | D835H | | | T417- |
| C759* | Q193- | F870- | D835V | | | Y418- |
| P763- | Q312- | W795R | D835Y | | | Y418S |
| L775* | T191- | S786L | D839G | | | |
| E824- | K304- | R771* | E573- | | | |
| E1006* | A184D | W753R | E596- | | | |
| | R300S | F752L | F590- | | | |
| | R178L | P743S | F594- | | | |
| | Y62* | R736C | G583- | | | |
| | H6Y | F732C | I836- | | | |
| | A113- | F731Y | K602- | | | |
| | G96- | N717I | L789- | | | |
| | A91- | D712Y | M578- | | | |
| | A79G | G707D | M837- | | | |
| | F77- | I705T | N587- | | | |
| | F73- | Q692- | N676K | | | |
| | P70- | V684- | Q577- | | | |
| | D69- | G673S | Q580- | | | |
| | G38- | S669- | R595- | | | |
| | A30- | S669F | S451F | | | |
| | P23- | P625- | S574- | | | |
| | Y7* | R597- | S584- | | | |
| | | R597P | S585- | | | |
| | | C586Y | T582- | | | |
| | | L566* | V581- | | | |
| | | G543C | V592- | | | |
| | | Q527* | W603- | | | |
| | | L508- | Y572- | | | |
| | | R379C | Y589- | | | |
| | | H355- | Y591- | | | |
| | | W330* | Y597- | | | |
| | | R326H | Y599- | | | |
| | | S304- | | | | |
| | | E30A | | | | |

| NPM1 | NRAS | SRSF2 | TET2 | TP53 | WT1 |
|---|---|---|---|---|---|
| L258- | G12A | P95H | A1355T | A6P | A113- |
| L287- | G12C | P95L | A1379V | C124- | H246- |
| W259- | G12D | P95R | A1837- | E310- | H441- |
| W261- | G12S | | A991- | G134R | P134- |
| W288- | G12V | | C1271- | K93- | P134Q |
| W290- | G13C | | C1289S | L72Q | P266- |
| | G13D | | C1298- | P190- | P376- |
| | G13R | | D1384N | P322- | R369- |
| | Q61H | | D1587- | Q4* | R370- |
| | Q61K | | D688- | R116Q | R380- |
| | Q61R | | E692* | R141C | R458* |
| | S17N | | G1137V | R141H | R462L |
| | Y64D | | G1275R | R150W | R462P |
| | | | G1288C | R26C | R462Q |
| | | | G1430- | R273H | S138- |
| | | | G1719- | R81* | S381- |
| | | | G1869W | S83- | S381* |
| | | | G563- | T86 | S381X |
| | | | G613- | V140M | T385- |
| | | | H1219D | V25F | V368- |
| | | | L1457I | V84- | V379- |
| | | | L1511- | V84G | |
| | | | M1456- | V84M | |
| | | | N1102- | Y102C | |
| | | | N1103- | Y73C | |
| | | | N1266S | Y88C | |
| | | | N1774- | | |
| | | | P1115- | | |
| | | | P408- | | |
| | | | P818- | | |
| | | | Q1021- | | |
| | | | Q1501* | | |
| | | | Q383* | | |
| | | | Q635* | | |
| | | | Q705* | | |
| | | | Q744* | | |
| | | | Q810* | | |
| | | | R1261C | | |
| | | | R1516* | | |
| | | | S1050* | | |
| | | | S1449- | | |
| | | | S1838A | | |
| | | | S217- | | |
| | | | S675* | | |
| | | | Y1598- | | |

*= mutation introducing a stop codon;
- = frameshift mutation
ASXL1 = NM_015338;
CEBPA = NM_004364;
DNMT3A = NM_022552;
FLT3 = NM_004119;
IDH1 = NM_005896;
IDH2 = NM_002168;
KIT = NM_001093772;
NPM1 = NM_002520;
NRAS = NM_002524;
RUNX1 = NM_001754;
SRSF2 = NM_003016;
TET2 = NM_017628;
TP53 = NM_001276760;
WT1 = NM_024426.

Notably, AML specimens with simultaneous mutations in NPM1, DNMT3A and FLT3-ITD, which was recently associated with a particularly adverse prognostic (Papaemmanuil, E. et al. N Engl J Med 374, 2209-2221, 2016), were shown to be particularly sensitive to Mubritinib (median EC₅₀ of 96 nM as compared to 423 nM in other AMLs subtypes, FIG. 3G). Focusing on specimens with a NK (representing 30% of AML patients, FIG. 3H) or on samples with a NK and mutations in NPM1 (representing 25% of AML patient samples, FIG. 3I), limiting dilution assays in immunocompromised mice demonstrate that Mubritinib-sensitive specimens have significantly higher frequencies of leukemic stem cells (LSCs) relative to Mubritinib-resistant samples. High LSC frequency has been shown to be generally associated with increased minimal residual disease (MRD) and poor prognosis (see, e.g., Moshaver B. et al., Stem Cells 26, 3059-3067 (2008)). Overall, these results provide useful indications for the selection of AML patient populations who might most benefit from treatment with Mubritinib, and provide evidence of a potential link between sensitivity to Mubritinib and the frequency of LSCs in patient samples.

### Example 5: Assessment of the Mechanism of Action of Mubritinib in AML

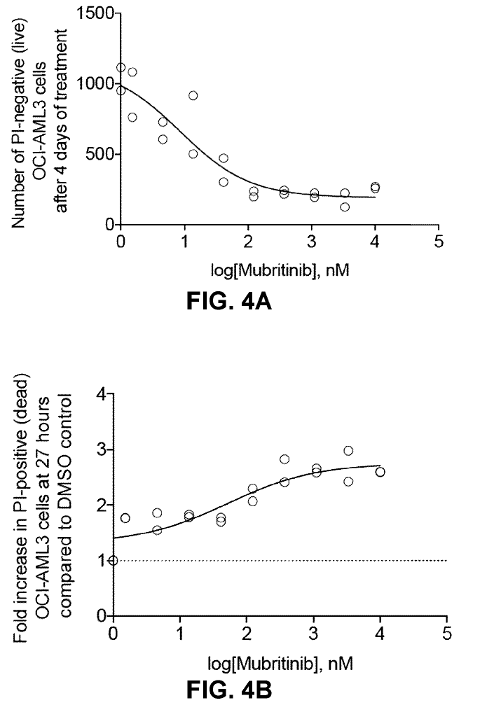

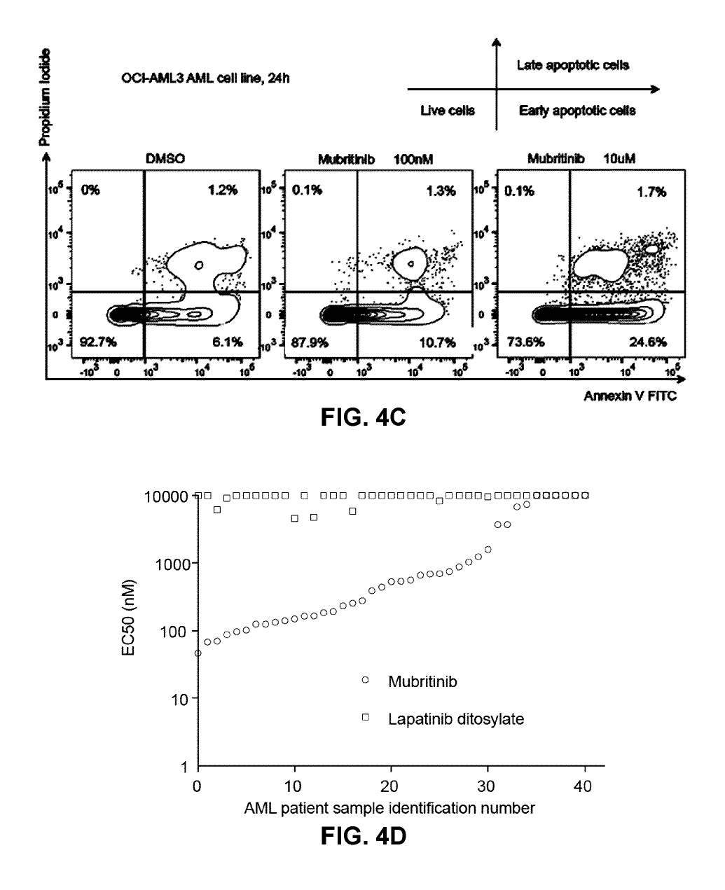

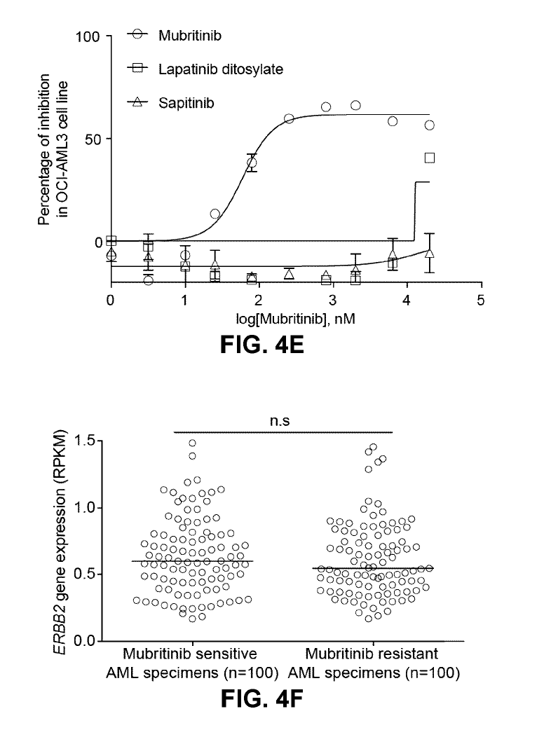

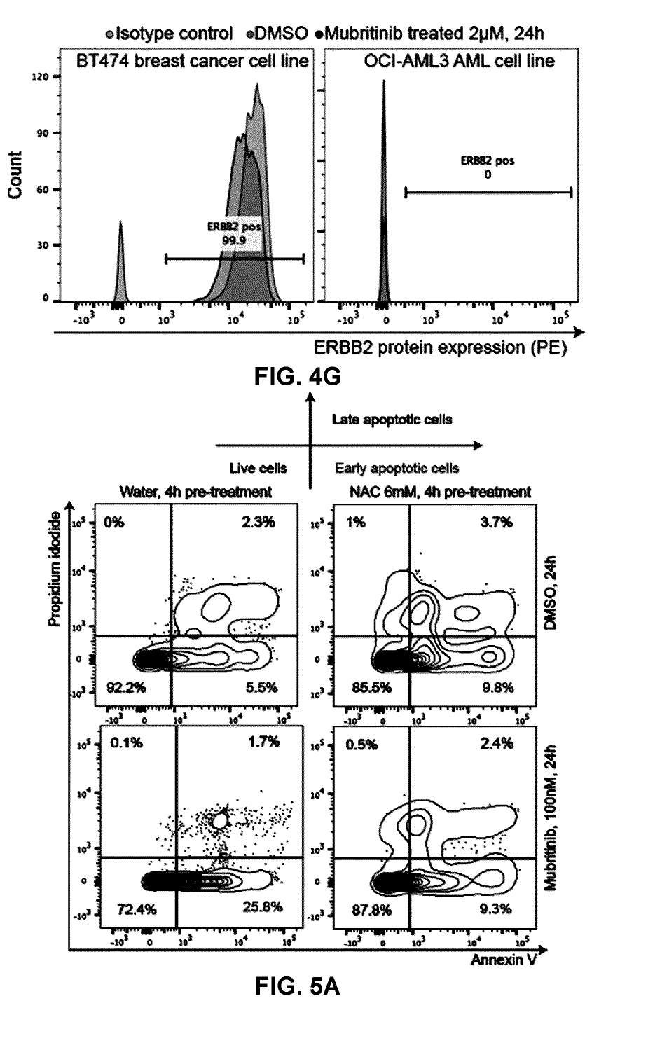

Mubritinib treatment induces a reduction of live cell counts (as measured by a decrease in Propidium Iodide (PI)-negative cells) in a dose-dependent manner (FIG. 4A). Cells are lost due to an increase in the number of dead (PI-positive) cells (FIG. 4B), which appear to die through apoptosis, as suggested by the increase in Annexin V staining (a marker of apoptosis) in a Mubritinib-sensitive AML cell line, OCI-AML3. (FIG. 4C).

Mubritinib is described as a specific ERBB2 inhibitor (Nagasawa, J. et al. Int J Urol 13, 587-592, 2006), and it was next assessed whether Mubritinib acts through this target on AML cells. Another potent ERBB2 inhibitor, Lapatinib, was included in the primary screen (FIG. 2A); strikingly, all 41 AML patient specimens tested were resistant to Lapatinib, in contrast to Mubritinib (FIG. 4D). Similarly, OCI-AML3 cells were sensitive to Mubritinib, but not to Lapatinib or Sapitinib, two other potent ERBB2 inhibitors (FIG. 4E), providing evidence that Mubritinib effects on AML cells is not shared by the class of ERBB2 inhibitors. Overall, AML patient cells that were sensitive to Mubritinib had the same low level of ERBB2 gene expression level as Mubritinib-resistant specimens (median around 0.5 RPKM, FIG. 4F). Finally, flow cytometry analysis of ERBB2 expression have demonstrated that the Mubritinib-sensitive AML cell line OCI-AML3 does not express detectable levels of ERBB2 protein, contrary to a positive control breast cancer cell line BT474 (FIG. 4G). Taken together, these results indicate that Mubritinib induces cell death in AML cells through a target other than ERBB2.

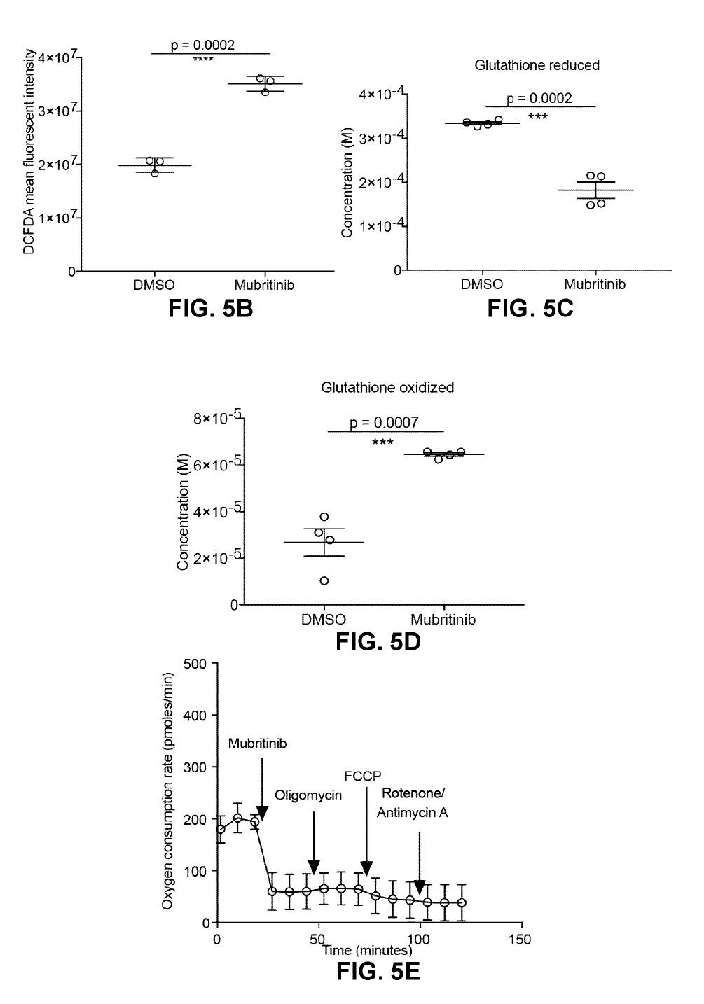

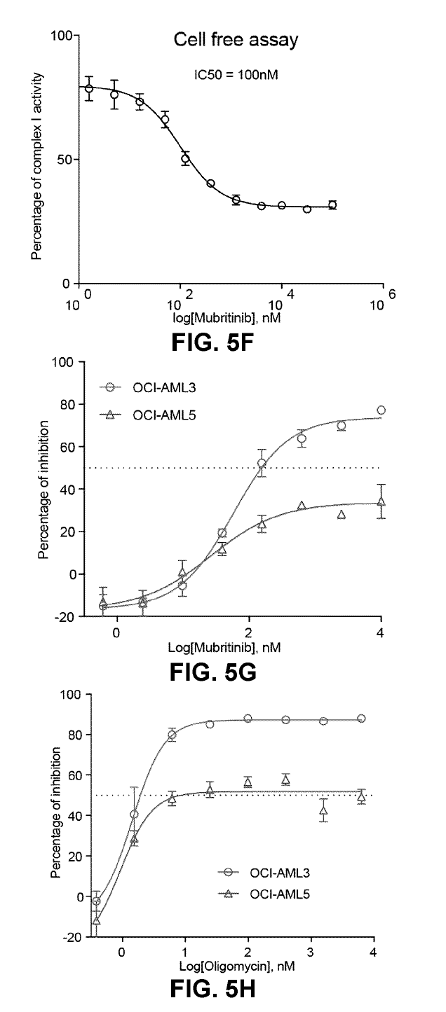

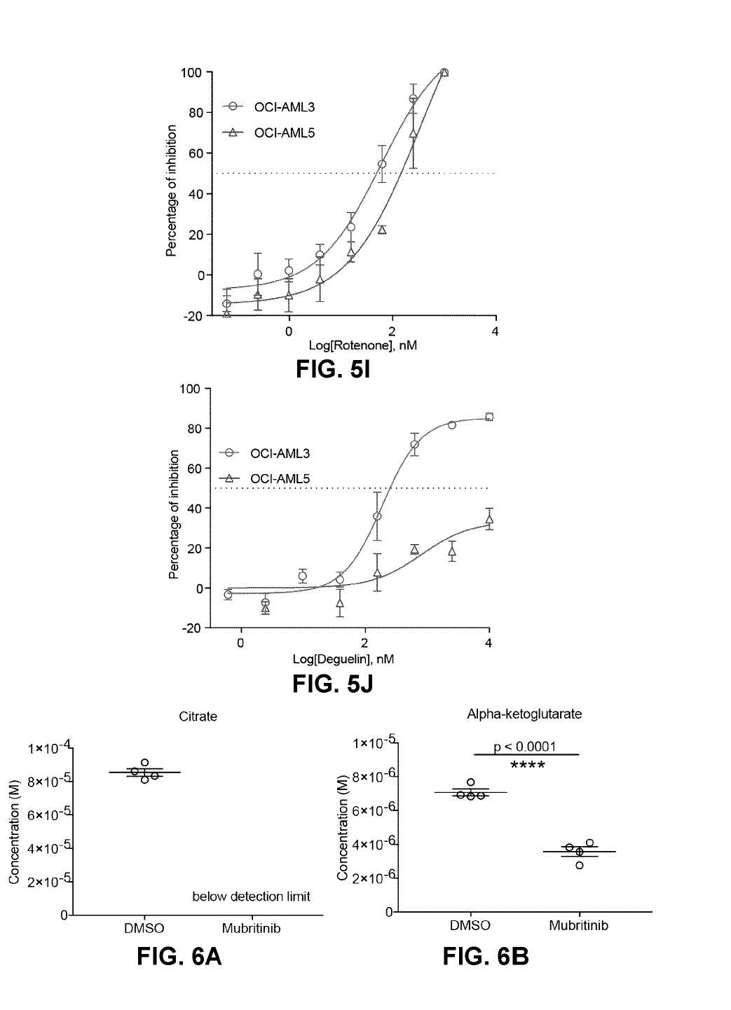

It was next assessed whether apoptosis induced by Mubritinib treatment involved oxidative stress, e.g., through the increase in reactive oxygen species (ROS). As shown in FIG. 5A, Mubritinib-induced apoptosis was significantly decreased when AML3 leukemic cells were incubated in the presence of the ROS scavenger N-acetyl-cysteine (NAC). Also, the level of ROS activity (hydroxyl, peroxyl), as assessed by the 2′,7′-dichlorofluorescin diacetate (DCFDA) fluorogenic dye, was increased in AML3 cells treated with Mubritinib (FIG. 5B). Also, upon Mubritinib treatment (500 nM, 24 h), the levels of oxidized glutathione were increased (FIG. 5C), while the levels of reduced glutathione were decreased (FIG. 5D), relative to DMSO control, in these leukemic cells, indicating that Mubritinib induces oxidative stress in sensitive leukemic cells.

To assess whether Mubritinib-induced oxidative stress in sensitive leukemic cells involves mitochondrial activity, namely the activity of the electron transfer chain (ETC), the oxygen consumption rate was measured in cells treated with Mubritinib. As shown in FIG. 5E, acute injection of Mubritinib (1 μM) in OCI-AML3 leukemic cells impaired oxygen consumption rate in these cells. The data presented in FIG. 5F demonstrates that Mubritinib exhibits an inhibitory effect on ETC Complex I activity. FIG. 5G shows that the leukemic cell line OCI-AML5 is resistant to Mubritinib-induced cell death, in contrast to OCI-AML3 cells, which suggests that these cell lines exhibit metabolic or mitochondrial function disparities. Consistent with this hypothesis, OCI-AML3 were shown to be more sensitive than OCI-AML5 to other mitochondrial activity inhibitors such as Oligomycin (inhibitor of the F₁-F₀ ATP synthase, complex V, FIG. 5H), Rotenone (Class A inhibitor of complex I, FIG. 5I) and Deguelin (Class A inhibitor of complex I, FIG. 5J).

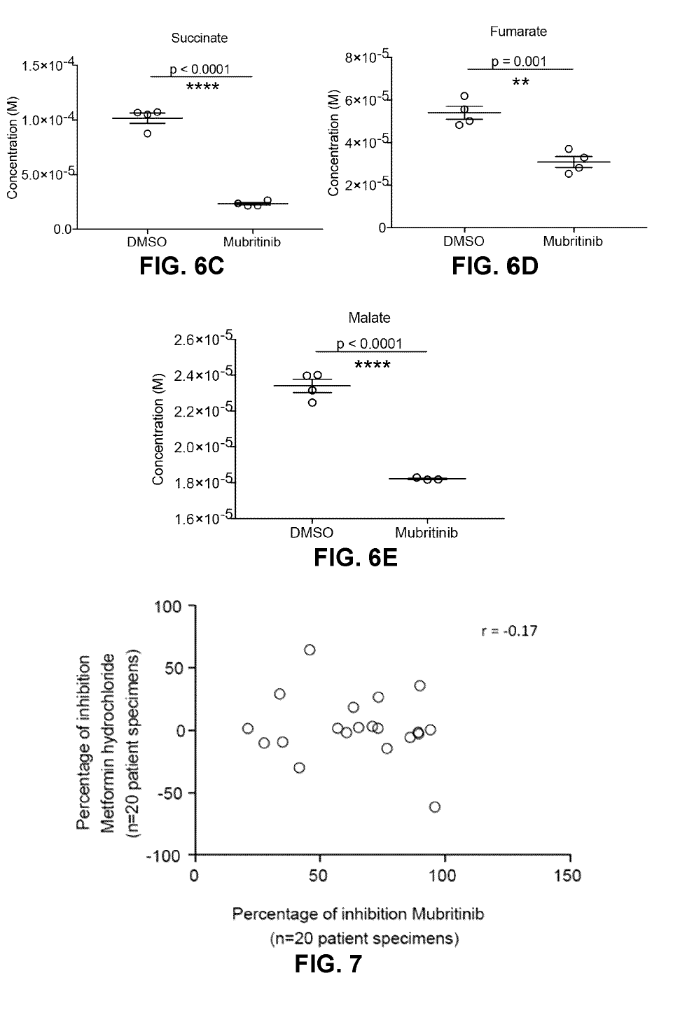

The results depicted in FIGS. 6A-6E show that Mubritinib treatment reduces the levels of several intermediates of the citric acid cycle in OCI-AML3 cells, namely citrate (FIG. 6A), alpha-ketoglutarate (FIG. 6B), succinate (FIG. 6C), fumarate (FIG. 6D), and malate (FIG. 6E), confirming that Mubritinib impairs mitochondrial activity/respiration in these leukemic cells.

The antidiabetic drug metformin has been shown to inhibit the proliferation of tumor cell lines (prostate, malignant melanoma and breast), and to synergistically sensitize FLT3-ITD+ AML cells to the kinase inhibitor sorafenib through enhancement of autophagy (Wang et al., 2015. Leukemia Research 39: 1421-1427). Metformin has also been shown to inhibit mitochondrial complex I in HCT116 p53^{-/-} colon cancer cells, but contrary to rotenone and antimycin, it does not increase ROS (H₂O₂) production by these cells (Wheaton et al., 2014. Elife. 13(3):e02242). It was thus next tested whether there was a correlation between Mubritinib and metformin effect on AML cells. As shown in FIG. 7, comparison of the percentage of AML cell inhibition induced by each compound throughout the 20 AML specimens tested shows a lack of correlation (Pearson correlation coefficient r=−0.17) between Mubritinib's and Metformin's inhibitory effects in leukemic cells, providing evidence that these two compounds act through different mechanisms of action and target different AML cells.

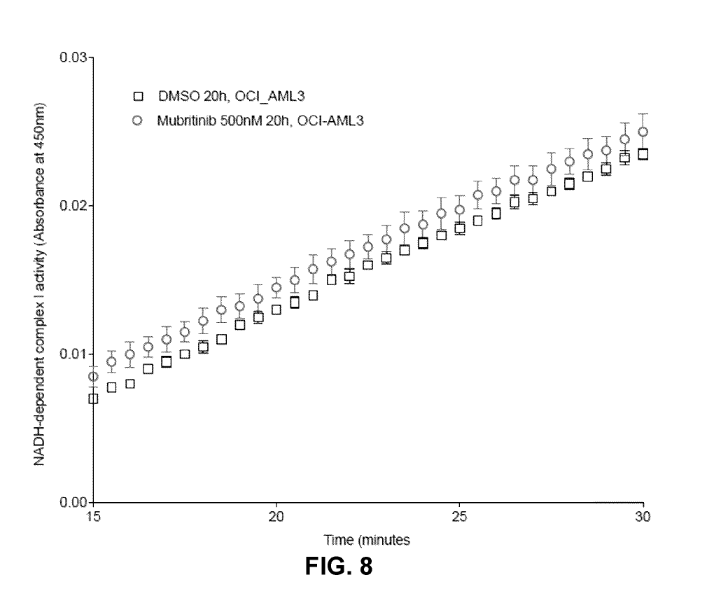

The inhibitory effect of Mubritinib on Complex I enzyme activity in OCI-AML3 cells was further assessed using the Complex I Enzyme Activity Microplate Assay Kit according to the manufacturer's instructions (Abcam, catalog No. ab109721). This assay measures the diaphorase-type activity of Complex I, which is not dependant on the presence of ubiquinone, and therefore inhibitors that bind at or near the ubiquinone binding site, such as rotenone, do not inhibit this assay. The results depicted in FIG. 8 show that Mubritinib's inhibitory effect on complex I is NADH-independent, as is described for class A inhibitors such as rotenone (Fato et al., Biochim Biophys Acta, 2009 May; 1787(5): 384-392), consistent with Mubritinib's ability to increase ROS in these cells.

Although the present invention has been described hereinabove by way of specific embodiments thereof, it can be modified, without departing from the spirit and nature of the subject invention as defined in the appended claims.

1. A method for treating acute myeloid leukemia (AML) in a subject in need thereof comprising administering to said subject an effective amount of a class A electron transport chain (ETC) complex I inhibitor.

2. (canceled)

3. The method of claim 1, wherein said class A ETC complex I inhibitor is Mubritinib or a pharmaceutically acceptable salt thereof.

4. The method of claim 1, wherein said AML is poor prognosis AML.

5. The method of claim 1, wherein said AML comprises at least one of the following features:
(a) high level of expression of one or more homeobox (HOX)-network genes;
(b) high level of expression of one or more of the genes depicted in Table 1;
(c) low level of expression of one or more of the genes depicted in Table 2;
(d) one or more of the following cytogenetic or molecular risk factor: intermediate cytogenetic risk, Normal Karyotype (NK), mutated NPM1, mutated CEBPA, mutated FLT3, mutated DNA methylation genes, mutated RUNX1, mutated WT1, mutated SRSF2, intermediate cytogenetic risk with abnormal karyotype (intern(abnK)), trisomy 8 (+8) and abnormal chr(5/7); and
(e) a leukemic stem cell (LSC) frequency of about 1 LSC per 1×10⁶ total cells, or more.

6. The method of claim 5, wherein said AML comprises high level of expression of one or more HOX-network genes.

7. The method of claim 6, wherein said one or more HOX-network genes are HOXB1, HOXB2, HOXB3, HOXB5, HOXB6, HOXB7, HOXB9, HOXB-AS3, HOXA1, HOXA2, HOXA3, HOXA4, HOXA5, HOXA6, HOXA7, HOXA9, HOXA10, HOXA10-AS, HOXA11, HOXA11-AS, MEIS1 and/or PBX3.

8. The method of claim 7, wherein said one or more HOX-network genes are HOXA9 and/or HOXA10.

9. The method of claim 5, wherein said AML comprises high level of expression of one or more of the genes depicted in Table 1.

10. The method of claim 5, wherein said AML comprises low level of expression of one or more of the genes depicted in Table 2.

11. The method of claim 5, wherein said AML comprises one or more of the cytogenetic or molecular risk factor defined in item (d) of claim 4.

12. The method of claim 5, wherein said AML is intermediate cytogenetic risk AML and/or NK-AML.

13. The method of claim 11, wherein said AML comprises at least two or three of said cytogenetic or molecular risk factors.

14. (canceled)

15. The method of claim 5, wherein said AML comprises a mutated NPM1, a mutated FLT3 and/or a mutated DNA methylation gene.

16. The method of claim 15, wherein said DNA methylation gene is DNMT3A or IDH1.

17. The method of claim 16, wherein said AML comprises a mutated NPM1, a mutated FLT3 and a mutated DNMT3A.

18-19. (canceled)

20. The method of claim 5, wherein said AML comprises an LSC frequency of about 1 LSC per 5×10⁵ total cells, or more.

21. The method of claim 5, wherein said AML comprises at least two or three of features (a) to (e) defined in claim 5.

22. (canceled)

23. The method of claim 5, wherein said AML is NK-AML with mutated NPM1.

24. The method of claim 1, wherein the class A ETC complex I inhibitor is present in a pharmaceutical composition.

25. The method of claim 1, wherein the subject is a pediatric subject or an adult subject.

26-37. (canceled)
